(19) United States
(12) Patent Application Publication
(10) Pub. No.: US 2020/0338334 A1
(43) Pub. Date: Oct. 29, 2020

(54) Fibrous Connective Tissue Healing System

(71) Applicant: Neurostim Technologies LLC, Waltham, MA (US)

(72) Inventors: Hoo-min D. TOONG, Cambridge, MA (US); William C. ALTMANN, Austin, TX (US); Michael Bernard DRUKE, Half Moon Bay, CA (US)

(21) Appl. No.: 16/924,692

(22) Filed: Jul. 9, 2020

Related U.S. Application Data
(63) Continuation-in-part of application No. 16/181,929, filed on Nov. 6, 2018.
Application No. 16/181,929 is a continuation-in-part of application No. 15/882,213, filed on Jan. 29, 2018.
(60) Provisional application No. 62/984,616, filed on Mar. 3, 2020.
(60) Provisional application No. 62/872,109, filed on Jul. 9, 2019.
(60) Provisional application No. 62/661,256, filed on Apr. 23, 2018.
(60) Provisional application No. 62/582,634, filed on Nov. 7, 2017.
(60) Provisional application No. 62/582,634, filed on Nov. 7, 2017.

(51) Int. Cl. A61N 1/04; A61N 1/36

(52) U.S. Cl. CPC A61N 1/0468; A61N 1/36034; A61N 1/36031; A61N 1/0476

(57) ABSTRACT

Example inventions heal damage to a fibrous connective tissue. Examples affix a patch externally on a dermis of a user adjacent to a damaged fibrous connective tissue of the user, the patch comprising a flexible substrate, a processor directly coupled to the substrate, and electrodes directly coupled to the substrate. Examples then activate the patch, the activating comprising generating an electrical stimuli via the electrodes that is directed to the damaged fibrous connective tissue.

### CROSS REFERENCE TO RELATED APPLICATIONS

This application claims priority to U.S. Provisional Patent Application Ser. No. 62/872,109, filed on Jul. 9, 2019, and to U.S. Provisional Patent Application Ser. No. 62/984,616, filed on Mar. 3, 2020, and claims priority as a continuation-in-part application to U.S. patent application Ser. No. 16/181,929, filed on Nov. 6, 2018, which claims priority to U.S. Provisional Patent Application Ser. No. 62/582,634, filed on Nov. 7, 2017, and to U.S. Provisional Patent Application Ser. No. 62/661,256, filed on Apr. 23, 2018, and claims priority as a continuation-in-part application of U.S. patent application Ser. No. 15/882,213, filed on Jan. 29, 2018, which claims priority to U.S. Provisional Patent Application Ser. No. 62/582,634, filed on Nov. 7, 2017. The disclosure of each of these applications is hereby incorporated by reference.

### FIELD

Example inventions are directed to systems and methods for reconstructing and healing damage to fibrous connective tissue, such as plantar fasciitis, an ankle sprain, or an Achilles tendon pull.

### BACKGROUND INFORMATION

Plantar fasciitis is a painful affliction which affects walking, standing and other motions which place weight on the affected heel. Plantar fasciitis is the inflammation of the plantar fascia in the heel of one or both feet. Plantar fasciitis also involves damage to the collagen fibers in the plantar fascia. Healing in the collagen fibers is slow due to the low blood flow in those structures.

### BRIEF DESCRIPTION OF THE DRAWINGS

FIG. 1 illustrates an example patch that is affixed to a location behind an ankle bone of a user.

FIG. 2 is a block diagram illustrating hardware/software related elements of an example of the patch of FIG. 1.

FIG. 3A is a circuit diagram of an example of a boosted voltage circuit that provides feedback.

FIG. 3B is a circuit diagram of an example of a charge application circuit that uses an output of the boosted voltage circuit.

FIG. 4 is a flow diagram of the functionality of the controller of monitoring and controlling the output voltage, including its ramp rate.

FIG. 5 is a flow diagram in accordance with one example of an adaptive protocol.

FIG. 6 is a Differential Integrator Circuit used in the adaptive protocol in accordance with one example.

FIG. 7 is a table relating charge duration vs. frequency to provide feedback to the adaptive protocol in accordance with one example.

FIG. 8 is an illustration of components of a fibrous connective tissue healing system in accordance with example inventions.

FIGS. 9A and 9B illustrate an ankle of a user.

FIG. 10 illustrates the connectivity of a patch and a smart controller with a data store, a network, and the cloud in example inventions.

FIG. 11 illustrates a feedback loop to create a closed-loop system between the user, patch and smart controller in example inventions.

FIG. 12 illustrates the anatomical structure of a tendon.

FIG. 13 is an illustration of components of a fibrous connective tissue reconstruction and healing system in accordance with example inventions.

FIG. 14 illustrates a healing patch conforming to the shape of the angle and sole of the foot in accordance with example inventions.

FIG. 15 illustrates a healing patch affixed to the interior side of a bandage in accordance with example inventions.

FIG. 16 illustrates several bandage arrangements with the patch in accordance with example inventions.

FIG. 17 illustrates the patch with multiple electrodes that are adapted to provide both stimulation and sensing in accordance with example inventions.

FIG. 18 illustrates a stack-up view of the patch accordance to example inventions.

### DETAILED DESCRIPTION

A non-invasive nerve patch/activator in accordance with various examples disclosed herein includes novel circuitry to adequately boost voltage to a required level and to maintain a substantially constant level of charge for nerve activation. Further, a feedback loop provides for an automatic determination and adaptation of the applied charge level. In example inventions, the patch is used to heal and reconstruct damage associated with fibrous connective tissue.

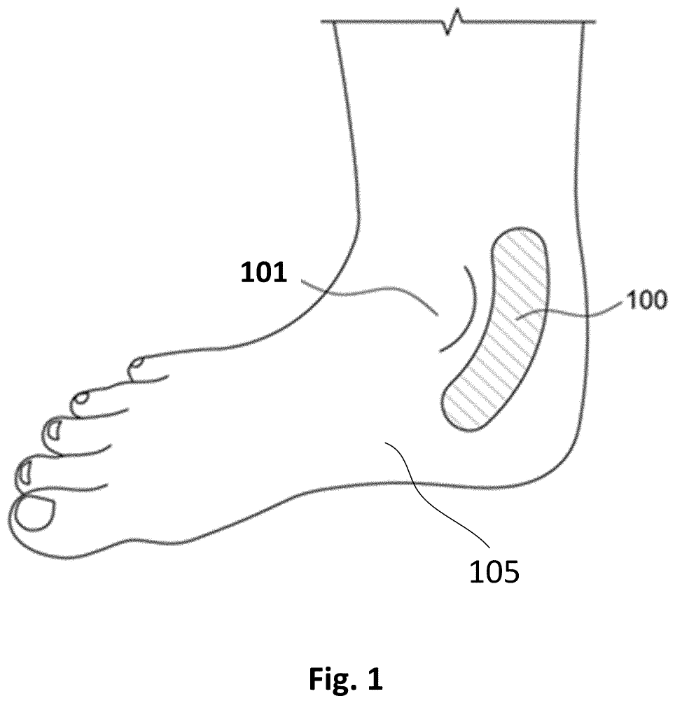

FIG. 1 illustrates an example patch 100, also referred to as a smart band aid or smartpad or Topical Nerve Activator (“TNA”) or topical nerve activation patch, that is affixed to a location behind an ankle bone 101 of a user 105 in one example use. In the example of FIG. 1, patch 100 is adapted to activate/stimulate the tibial nerve of user 105. In other examples, patch 100 is worn at different locations of user 105 to activate the tibial nerve from a different location, or to activate a different nerve of user 105.

Patch 100 is used to stimulate these nerves and is convenient, unobtrusive, self-powered, and may be controlled from a smartphone or other control device. This has the advantage of being non-invasive, controlled by consumers themselves, and potentially distributed over the counter without a prescription. Patch 100 provides a means of stimulating nerves without penetrating the dermis, and can be applied to the surface of the dermis at a location appropriate for the nerves of interest. In examples, patch 100 is applied by the user and is disposable.

Patch 100 in examples can be any type of device that can be fixedly attached to a user, using adhesive in some examples, and includes a processor/controller and instructions that are executed by the processor, or a hardware implementation without software instructions, as well as electrodes that apply an electrical stimulation to the surface of the user's skin, and associated electrical circuitry. Patch 100 in one example provides topical nerve activation/stimulation on the user to provide benefits to the user, including bladder management for an overactive bladder (“OAB”) or healing and reconstruction of fibrous connective tissue damage.

Patch 100 in one example can include a flexible substrate, a malleable dermis conforming bottom surface of the substrate including adhesive and adapted to contact the dermis, a flexible top outer surface of the substrate approximately parallel to the bottom surface, one or more electrodes positioned on the patch proximal to the bottom surface and located beneath the top outer surface and directly contacting the flexible substrate, electronic circuitry (as disclosed herein) embedded in the patch and located beneath the top outer surface and integrated as a system on a chip that is directly contacting the flexible substrate, the electronic circuitry integrated as a system on a chip and including an electrical signal generator integral to the malleable dermis conforming bottom surface configured to electrically activate the one or more electrodes, a signal activator coupled to the electrical signal generator, a nerve stimulation sensor that provides feedback in response to a stimulation of one or more nerves, an antenna configured to communicate with a remote activation device, a power source in electrical communication with the electrical signal generator, and the signal activator, where the signal activator is configured to activate in response to receipt of a communication with the activation device by the antenna and the electrical signal generator configured to generate one or more electrical stimuli in response to activation by the signal activator, and the electrical stimuli configured to stimulate one or more nerves of a user wearing patch 100 at least at one location proximate to patch 100. Additional details of examples of patch 100 beyond the novel details disclosed herein are disclosed in U.S. Pat. No. 10,016,600, entitled “Topical Neurological Stimulation”, the disclosure of which is hereby incorporated by reference.

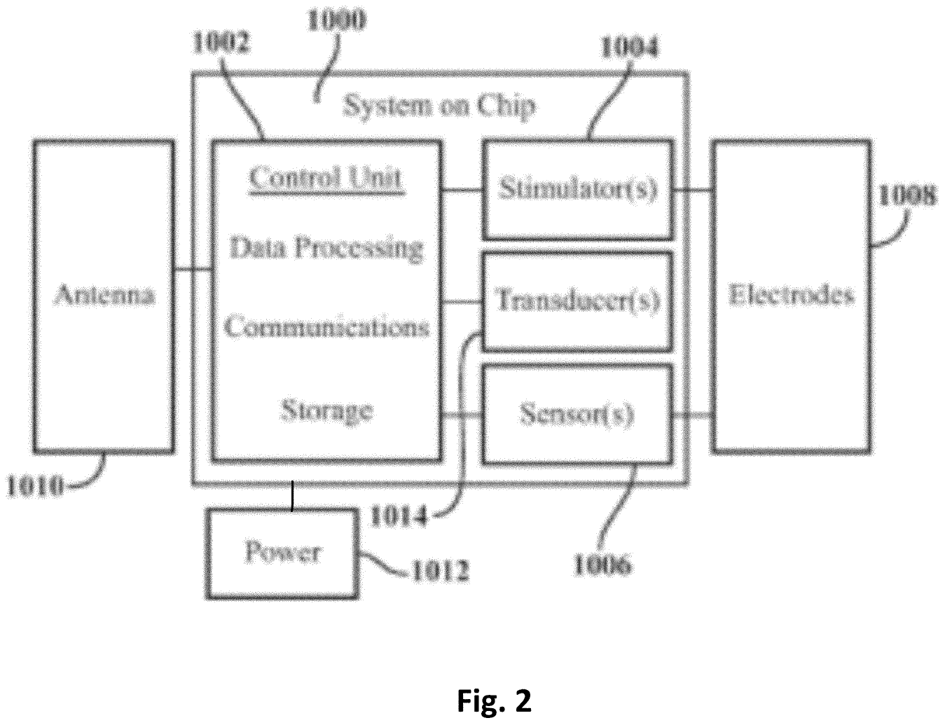

FIG. 2 is a block diagram illustrating hardware/software related elements of an example of patch 100 of FIG. 1. Patch 100 includes electronic circuits or chips 1000 that perform the functions of: communications with an external control device, such as a smartphone or fob, or external processing such as cloud based processing devices, nerve activation via electrodes 1008 that produce a wide range of electric fields according to a treatment regimen, and a wide range of sensors 1006 such as, but not limited to, mechanical motion and pressure, temperature, humidity, acoustic, chemical and positioning sensors. In another example, patch 100 includes transducers 1014 to transmit signals to the tissue or to receive signals from the tissue.

One arrangement is to integrate a wide variety of these functions into a system on a chip 1000. Within this is shown a control unit 1002 for data processing, communications, transducer interface and storage, and one or more stimulators 1004 and sensors 1006 that are connected to electrodes 1008. Control unit 1002 can be implemented by a general purpose processor/controller, or a specific purpose processor/controller, or a special purpose logical circuit. An antenna 1010 is incorporated for external communications by control unit 1002. Also included is an internal power supply 1012, which may be, for example, a battery. Other examples may include an external power supply. It may be necessary to include more than one chip to accommodate a wide range of voltages for data processing and stimulation. Electronic circuits and chips will communicate with each other via conductive tracks within the device capable of transferring data and/or power.

Patch 100 interprets a data stream from control unit 1002 to separate out message headers and delimiters from control instructions. In one example, control instructions include information such as voltage level and pulse pattern. Patch 100 activates stimulator 1004 to generate a stimulation signal to electrodes 1008 placed on the tissue according to the control instructions. In another example, patch 100 activates transducer 1014 to send a signal to the tissue. In another example, control instructions cause information such as voltage level and a pulse pattern to be retrieved from a library stored by patch 100, such as storage within control unit 1002.

Patch 100 receives sensory signals from the tissue and translates them to a data stream that is recognized by control unit 1002. Sensory signals can include electrical, mechanical, acoustic, optical and chemical signals. Sensory signals are received by patch 100 through electrodes 1008 or from other inputs originating from mechanical, acoustic, optical, or chemical transducers. For example, an electrical signal from the tissue is introduced to patch 100 through electrodes 1008, is converted from an analog signal to a digital signal and then inserted into a data stream that is sent through antenna 1010 to the external control device. In another example an acoustic signal is received by transducer 1014, converted from an analog signal to a digital signal and then inserted into a data stream that is sent through the antenna 1010 to the external control device. In some examples, sensory signals from the tissue are directly interfaced to the external control device for processing.

In examples of patch 100 disclosed above, when being used for therapeutic treatment such as bladder management for OAB or fibrous connective tissue damage healing and reconstruction, there is a need to control the voltage by boosting the voltage to a selected level and providing the same level of charge upon activation to a mammalian nerve. Further, there is a need to conserve battery life by selectively using battery power. Further, there is a need to create a compact electronics package to facilitate mounting the electronics package on a relatively small mammalian dermal patch in the range of the size of an ordinary band aid.

Adaptive Circuit

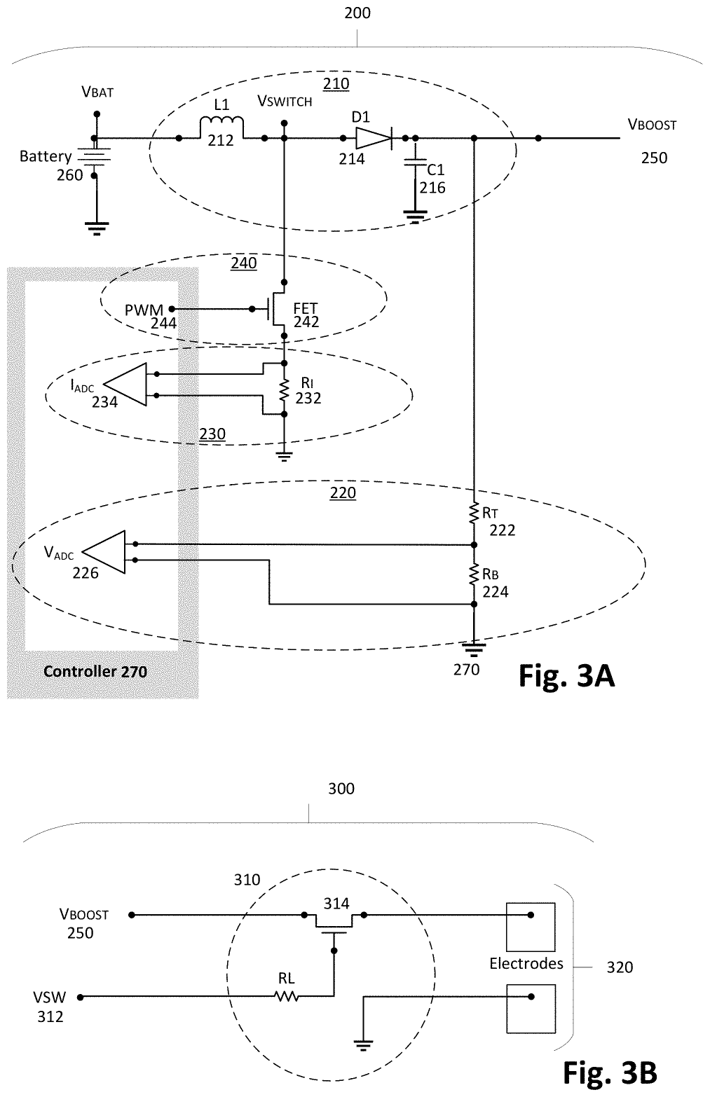

To meet the above needs, examples implement a novel boosted voltage circuit that includes a feedback circuit and a charge application circuit. FIG. 3A is a circuit diagram of an example of the boosted voltage circuit 200 that provides feedback. FIG. 3B is a circuit diagram of an example of a charge application circuit 300 that uses an output of boosted voltage circuit 200. Boosted voltage circuit 200 includes both electrical components and a controller/processor 270 that includes a sequence of instructions that together modify the voltage level of activation/stimulation delivered to the external dermis of user 105 by patch 100 through electrodes. Controller/processor 270 in examples implements control unit 1002 of FIG. 2.

Boosted voltage circuit 200 can replace an independent analog-controlled boost regulator by using a digital control loop to create a regulated voltage, output voltage 250, from the battery source. Output voltage 250 is provided as an input voltage to charge application circuit 300. In examples, this voltage provides nerve stimulation currents through the dermis/skin to deliver therapy for an overactive bladder. Output voltage 250, or “V_{Boost}”, at voltage output node 250, uses two digital feedback paths 220, 230, through controller 270. In each of these paths, controller 270 uses sequences of instructions to interpret the measured voltages at voltage monitor 226, or “V_{ADC}” and current monitor 234, or “I_{ADC}”, and determines the proper output control for accurate and stable output voltage 250.

Boosted voltage circuit 200 includes an inductor 212, a diode 214, a capacitor 216 that together implement a boosted converter circuit 210. A voltage monitoring circuit 220 includes a resistor divider formed by a top resistor 222, or “R_{T}”, a bottom resistor 224, or “R_{B}” and voltage monitor 226. A current monitoring circuit 230 includes a current measuring resistor 232, or “R_{I}” and current monitor 234. A pulse width modulation (“PWM”) circuit 240 includes a field-effect transistor (“FET”) switch 242, and a PWM driver 244. Output voltage 250 functions as a sink for the electrical energy. An input voltage 260, or “V_{BAT}”, is the source for the electrical energy, and can be implemented by power 1012 of FIG. 2.

PWM circuit 240 alters the “on” time within a digital square wave, fixed frequency signal to change the ratio of time that a power switch is commanded to be “on” versus “off.” In boosted voltage circuit 200, PWM driver 244 drives FET switch 242 to “on” and “off” states.

In operation, when FET switch 242 is on, i.e., conducting, the drain of FET switch 242 is brought down to Ground/GND or ground node 270. FET switch 242 remains on until its current reaches a level selected by controller 270 acting as a servo controller. This current is measured as a representative voltage on current measuring resistor 232 detected by current monitor 234. Due to the inductance of inductor 212, energy is stored in the magnetic field within inductor 212. The current flows through current measuring resistor 232 to ground until FET switch 242 is opened by PWM driver 244.

When the intended pulse width duration is achieved, controller 270 turns off FET switch 242. The current in inductor 212 reroutes from FET switch 242 to diode 214, causing diode 214 to forward current. Diode 214 charges capacitor 216. Therefore, the voltage level at capacitor 216 is controlled by controller 270.

Output voltage 250 is controlled using an outer servo loop of voltage monitor 226 and controller 270. Output voltage 250 is measured by the resistor divider using top resistor 222, bottom resistor 224, and voltage monitor 226. The values of top resistor 222 and bottom resistor 224 are selected to keep the voltage across bottom resistor 224 within the monitoring range of voltage monitor 226. Controller 270 monitors the output value from voltage monitor 226.

Charge application circuit 300 includes a pulse application circuit 310 that includes an enable switch 314. Controller 270 does not allow enable switch 314 to turn on unless output voltage 250 is within a desired upper and lower range of the desired value of output voltage 250. Pulse application circuit 310 is operated by controller 270 by asserting an enable signal 312, or “VSW”, which turns on enable switch 314 to pass the electrical energy represented by output voltage 250 through electrodes 320. At the same time, controller 270 continues to monitor output voltage 250 and controls PWM driver 244 to switch FET switch 242 on and off and to maintain capacitor 216 to the desired value of output voltage 250.

The stability of output voltage 250 can be increased by an optional inner feedback loop through FET Switch 242, current measuring resistor 232, and current monitor 234. Controller 270 monitors the output value from current monitor 234 at a faster rate than the monitoring on voltage monitor 226 so that the variations in the voltages achieved at the cathode of diode 214 are minimized, thereby improving control of the voltage swing and load sensitivity of output voltage 250.

In one example, a voltage doubler circuit is added to boosted voltage circuit 200 to double the high voltage output or to reduce voltage stress on FET 242. The voltage doubler circuit builds charge in a transfer capacitor when FET 242 is turned on and adds voltage to the output of boosted voltage circuit 200 when FET 242 is turned off.

As described, in examples, controller 270 uses multiple feedback loops to adjust the duty cycle of PWM driver 244 to create a stable output voltage 250 across a range of values. Controller 270 uses multiple feedback loops and monitoring circuit parameters to control output voltage 250 and to evaluate a proper function of the hardware. Controller 270 acts on the feedback and monitoring values in order to provide improved patient safety and reduced electrical hazard by disabling incorrect electrical functions.

In some examples, controller 270 implements the monitoring instructions in firmware or software code. In some examples, controller 270 implements the monitoring instructions in a hardware state machine.

In some examples, voltage monitor 226 is an internal feature of controller 270. In some examples, voltage monitor 226 is an external component, which delivers its digital output value to a digital input port of controller 270.

In some examples, current monitor 234 is an internal feature of controller 270. In some examples, current monitor 234 is an external component, which delivers its digital output value to a digital input port of controller 270.

An advantage of boosted voltage circuit 200 over known circuits is decreased component count which may result in reduced costs, reduced circuit board size and higher reliability. Further, boosted voltage circuit 200 provides for centralized processing of all feedback data which leads to faster response to malfunctions. Further, boosted voltage circuit 200 controls outflow current from V_{BAT} 260, which increases the battery's lifetime and reliability.

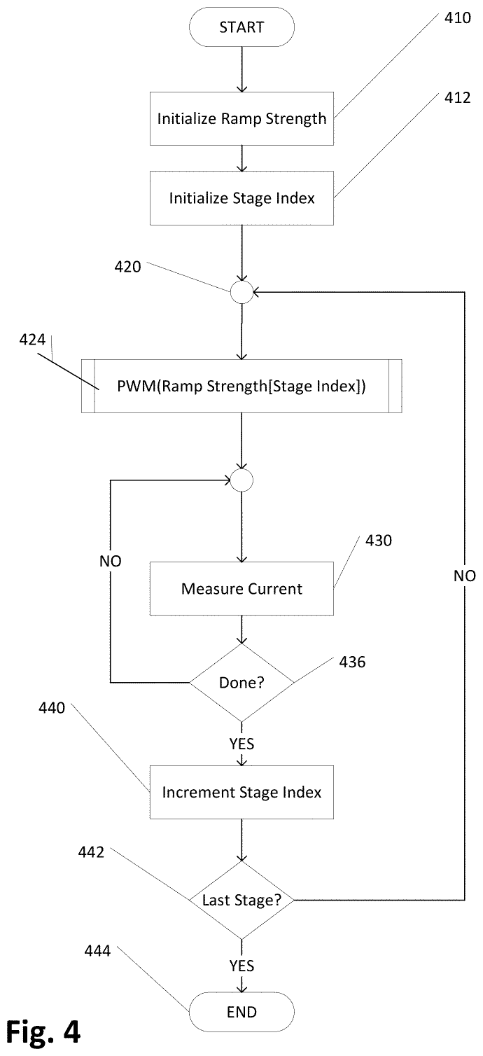

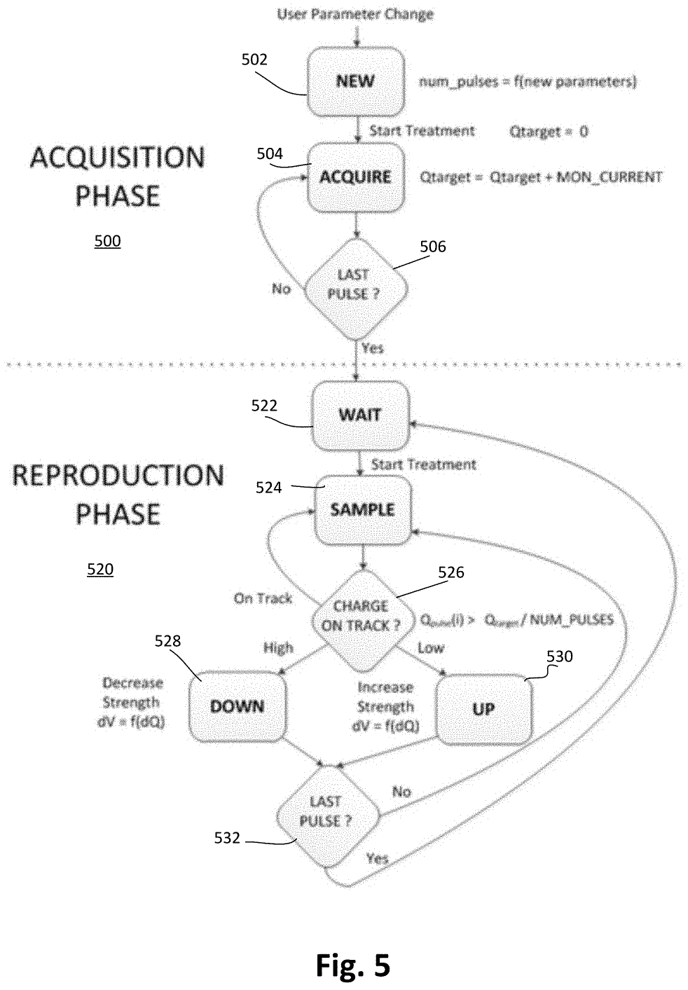

FIG. 4 is a flow diagram of the functionality of controller 270 of monitoring and controlling output voltage 250, including its ramp rate. In one example, the functionality of the flow diagram of FIG. 4, and FIG. 5 below, is implemented by software stored in memory or other computer readable or tangible medium, and executed by a processor. In other examples, the functionality may be performed by hardware (e.g., through the use of an application-specific integrated circuit (“ASIC”), a programmable gate array (“PGA”), a field programmable gate array (“FPGA”), etc.), or any combination of hardware and software.

The pulse width modulation of FET switch 242 is controlled by one or more pulses for which the setting of each pulse width allows more or less charge to accumulate as a voltage at capacitor 216 through diode 214. This pulse width setting is referred to as the ramp strength and it is initialized at 410. Controller 270 enables each pulse group in sequence with a pre-determined pulse width, one stage at a time, using a stage index that is initialized at 412. The desired ramp strength is converted to a pulse width at 424, which enables and disables FET switch 242 according to the pulse width. During the intervals when FET switch 242 is “on”, the current is measured by current monitor 234 at 430 and checked against the expected value at 436. When the current reaches the expected value, the stage is complete and the stage index is incremented at 440. If the desired number of stages have been applied 442, then the functionality is complete. Otherwise, the functionality continues to the next stage at 420.

As a result of the functionality of FIG. 4, V_{BAT} 260 used in patch 100 operates for longer periods as the current drawn from the battery ramps at a low rate of increase to reduce the peak current needed to achieve the final voltage level 250 for each activation/stimulation treatment. PWM 244 duty cycle is adjusted by controller 270 to change the ramp strength at 410 to improve the useful life of the battery.

An open loop protocol to control current to electrodes in known neural stimulation devices does not have feedback controls. It commands a voltage to be set, but does not check the actual current delivered. A stimulation pulse is sent based on preset parameters and cannot be modified based on feedback from the patient's anatomy. When the device is removed and repositioned, the electrode placement varies. Also the humidity and temperature of the anatomy changes throughout the day. All these factors affect the actual charge delivery if the voltage is preset. Charge control is a patient safety feature and facilitates an improvement in patient comfort, treatment consistency and efficacy of treatment.

In contrast, examples of patch 100 includes features that address these shortcomings using controller 270 to regulate the charge applied by electrodes 320. Controller 270 samples the voltage of the stimulation waveform, providing feedback and impedance calculations for an adaptive protocol to modify the stimulation waveform in real time. The current delivered to the anatomy by the stimulation waveform is integrated using a differential integrator and sampled and then summed to determine the actual charge delivered to the user for a treatment, such as OAB treatment. After every pulse in a stimulation event, this data is analyzed and used to modify, in real time, subsequent pulses.

This hardware adaptation allows a firmware protocol to implement the adaptive protocol. This protocol regulates the charge applied to the body by changing output voltage (“V_{BOOST}”) 250. A treatment is performed by a sequence of periodic pulses, which deliver charge into the body through electrodes 320. Some of the parameters of the treatment are fixed and some are user adjustable. The strength, duration and frequency may be user adjustable. The user may adjust these parameters as necessary for comfort and efficacy. The strength may be lowered if there is discomfort and raised if nothing is felt. The duration can be increased if the maximum acceptable strength results in an ineffective treatment.

Adaptive Protocol

A flow diagram in accordance with one example of the adaptive protocol disclosed above is shown in FIG. 5. The adaptive protocol strives to repeatedly and reliably deliver a target charge (“Q_{target}”) during a treatment and to account for any environmental changes. Therefore, the functionality of FIG. 5 is to adjust the charge level applied to a user based on feedback, rather than use a constant level.

The mathematical expression of this protocol is as follows:

Q_{target}=Q_{target}(A*dS+B*dT),

where A is the Strength Coefficient−determined empirically, dS is the user change in Strength, B is the Duration Coefficient−determined empirically, and dT is the user change in Duration.

The adaptive protocol includes two phases in one example: Acquisition phase 500 and Reproduction phase 520. Any change in user parameters places the adaptive protocol in the Acquisition phase. When the first treatment is started, a new baseline charge is computed based on the new parameters. At a new acquisition phase at 502, all data from the previous charge application is discarded. In one example, 502 indicates the first time for the current usage where the user places patch 100 on a portion of the body and manually adjusts the charge level, which is a series of charge pulses, until it feels suitable, or any time the charge level is changed, either manually or automatically. The treatment then starts. The mathematical expression of this function of the application of a charge is as follows:

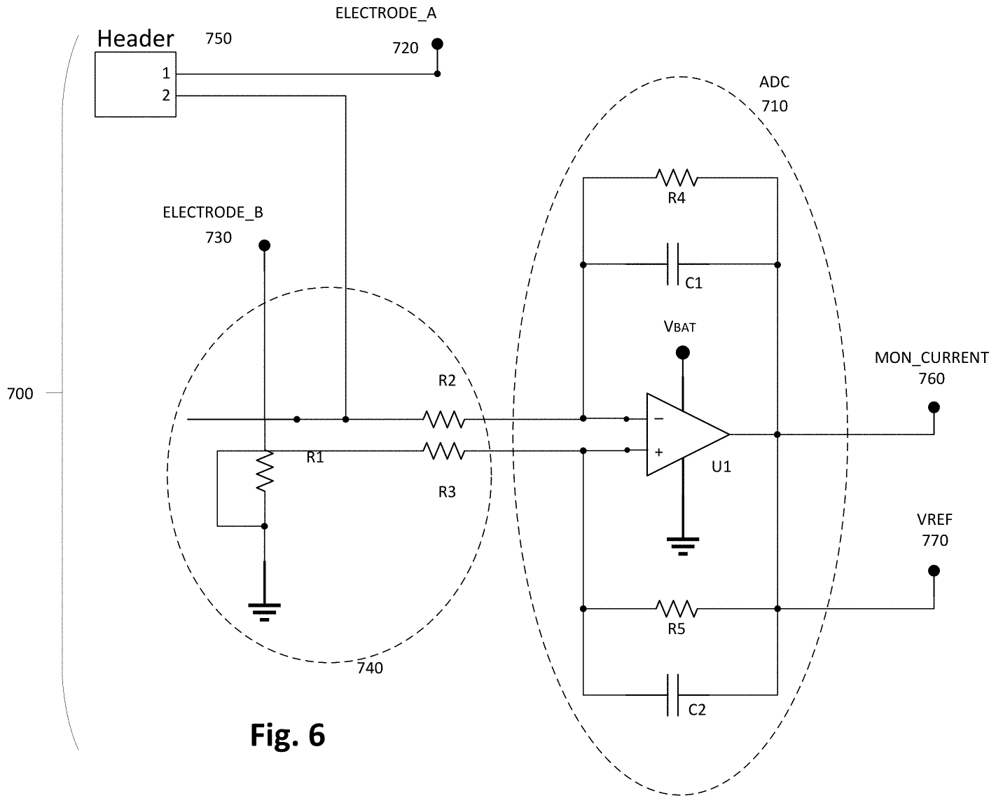

The charge delivered in a treatment is

$${Q}_{target} = {\sum }_{i = 1}^{T * f} {Q}_{pulse} ( i )$$

Where T is the duration; f is the frequency of “Rep Rate”; Qₚᵤₗₛₑ (i) is the measured charge delivered by Pulse (i) in the treatment pulse train provided as a voltage MON_CURRENT that is the result of a Differential Integrator circuit shown in FIG. 6 (i.e., the average amount of charge per pulse). Differential Integrator circuit 700 of FIG. 6 is an example of a circuit used to integrate current measured over time and quantify the delivered charge and therefore determine the charge output over a treatment pulse. The number of pulses in the treatment is T*f.

As shown in of FIG. 6, MON_CURRENT 760 is the result of the Differential Integrator Circuit 700. The Analog to Digital Conversion (“ADC”) 710 feature is used to quantify voltage into a number representing the delivered charge. The voltage is measured between Electrode A 720 and Electrode B 730, using a Kelvin Connection 740. Electrode A 720 and Electrode B 730 are connected to a header 750. A reference voltage, VREF 770, is included to keep the measurement in range.

In some examples, Analog to Digital Conversion 710 is an internal feature of controller 270. In some examples, Analog to Digital Conversion 710 is an external component, which delivers its digital output value to a digital input port on Controller 270.

At 504 and 506, every pulse is sampled. In one example, the functionality of 504 and 506 lasts for 10 seconds with a pulse rate of 20 Hz, which can be considered a full treatment cycle. The result of Acquisition phase 500 is the target pulse charge of Q_{target}.

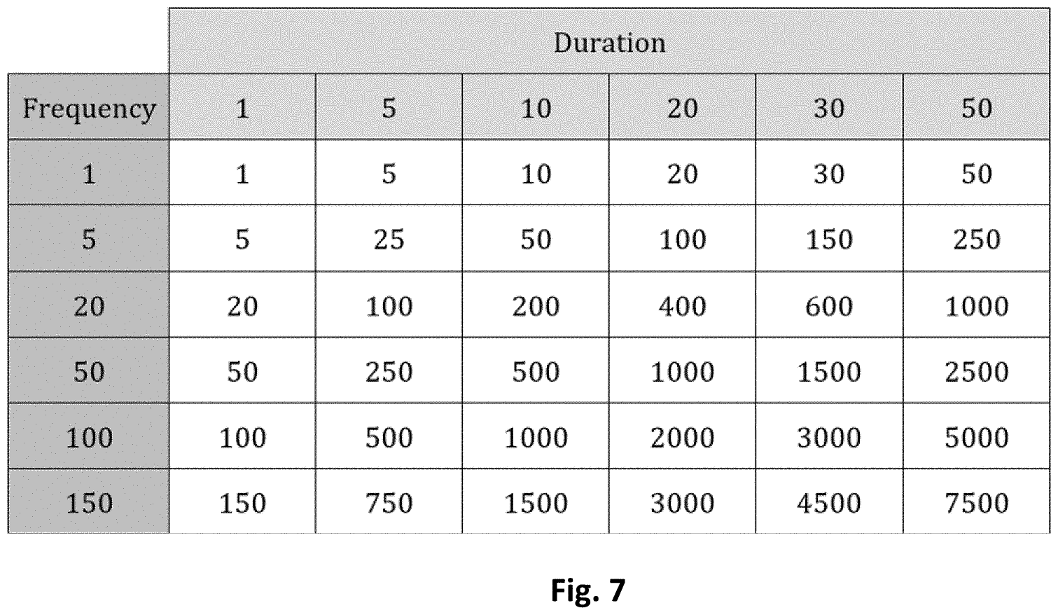

FIG. 7 is a table in accordance with one example showing the number of pulses per treatment measured against two parameters, frequency and duration. Frequency is shown on the Y-axis and duration on the X-axis. The adaptive protocol in general performs better when using more pulses. One example uses a minimum of 100 pulses to provide for solid convergence of charge data feedback, although a less number of pulses can be used in other examples. Referring to the FIG. 7, a frequency setting of 20 Hz and duration of 10 seconds produces 200 pulses.

The reproduction phase 520 begins in one example when the user initiates another subsequent treatment after acquisition phase 500 and the resulting acquisition of the baseline charge, Q_{target}. For example, a full treatment cycle, as discussed above, may take 10 seconds. After, for example, a two-hour pause as shown at wait period 522, the user may then initiate another treatment. During this phase, the adaptive protocol attempts to deliver Q_{target} for each subsequent treatment. The functionality of reproduction phase 520 is needed because, during the wait period 522, conditions such as the impedance of the user's body due to sweat or air humidity may have changed. The differential integrator is sampled at the end of each Pulse in the Treatment. At that point, the next treatment is started and the differential integrator is sampled for each pulse at 524 for purposes of comparison to the acquisition phase Q_{target}. Sampling the pulse includes measuring the output of the pulse in terms of total electric charge. The output of the integrator of FIG. 6 in voltage, referred to as Mon_Current 760, is a direct linear relationship to the delivered charge and provides a reading of how much charge is leaving the device and entering the user. At 526, each single pulse is compared to the charge value determined in Acquisition phase 500 (i.e., the target charge) and the next pulse will be adjusted in the direction of the difference.

NUM_PULSES=(T*f)

After each pulse, the observed charge, Qₚᵤₗₛₑ (i), is compared to the expected charge per pulse.

Qₚᵤₗₛₑ(i)>Q_{target}/NUM_PULSES?

The output charge or “V_{BOOST}” is then modified at either 528 (decreasing) or 530 (increasing) for the subsequent pulse by:

dV(i)=G[Q_{target}/NUM_PULSES−Qₚᵤₗₛₑ(i)]

where G is the Voltage adjustment Coefficient−determined empirically. The process continues until the last pulse at 532.

A safety feature assures that the V_{BOOST} will never be adjusted higher by more than 10%. If more charge is necessary, then the repetition rate or duration can be increased.

In one example a boosted voltage circuit uses dedicated circuits to servo the boosted voltage. These circuits process voltage and/or current measurements to control the PWM duty cycle of the boosted voltage circuit's switch. The system controller can set the voltage by adjusting the gain of the feedback loop in the boosted voltage circuit. This is done with a digital potentiometer or other digital to analog circuit.

In one example, in general, the current is sampled for every pulse during acquisition phase 500 to establish target charge for reproduction. The voltage is then adjusted via a digital potentiometer, herein referred to as “Pot”, during reproduction phase 520 to achieve the established target_charge.

The digital Pot is calibrated with the actual voltage at startup. A table is generated with sampled voltage for each wiper value. Tables are also precomputed storing the Pot wiper increment needed for 1 v and 5 v output delta at each pot level. This enables quick reference for voltage adjustments during the reproduction phase. The tables may need periodic recalibration due to battery level.

In one example, during acquisition phase 500, the data set=100 pulses and every pulse is sampled and the average is used as the target_charge for reproduction phase 520. In general, fewer pulses provide a weaker data sample to use as a basis for reproduction phase 520.

In one example, during acquisition phase 500, the maximum data set=1000 pulses. The maximum is used to avoid overflow of 32 bit integers in accumulating the sum of samples. Further, 1000 pulses in one example is a sufficiently large data set and collecting more is likely unnecessary.

After 1000 pulses for the above example, the target_charge is computed. Additional pulses beyond 1000 in the acquisition phase do not contribute to the computation of the target charge. In other examples, the maximum data set is greater than 1000 pulses when longer treatment cycle times are desired.

In one example, the first 3-4 pulses are generally higher than the rest so these are not used in acquisition phase 500. This is also accounted for in reproduction phase 520. Using these too high values can result in target charge being set too high and over stimulating on the subsequent treatments in reproduction phase 520. In other examples, more advanced averaging algorithms could be applied to eliminate high and low values.

In an example, there may be a safety concern about automatically increasing the voltage. For example, if there is poor connection between the device and the user's skin, the voltage may auto-adjust at 530 up to the max. The impedance may then be reduced, for example by the user pressing the device firmly, which may result in a sudden high current. Therefore, in one example, if the sample is 500 mv or more higher than the target, it immediately adjusts to the minimum voltage. This example then remains in reproduction phase 520 and should adjust back to the target current/charge level. In another example, the maximum voltage increase is set for a single treatment (e.g., 10V). More than that is not needed to achieve the established target_charge. In another example, a max is set for V_{BOOST} (e.g., 80V).

In various examples, it is desired to have stability during reproduction phase 520. In one example, this is accomplished by adjusting the voltage by steps. However, a relatively large step adjustment can result in oscillation or over stimulation. Therefore, voltage adjustments may be made in smaller steps. The step size may be based on both the delta between the target and sample current as well as on the actual V_{BOOST} voltage level. This facilitates a quick and stable/smooth convergence to the target charge and uses a more gradual adjustments at lower voltages for more sensitive users.

The following are the conditions that may be evaluated to determine the adjustment step.

delta-mon_current = abs(sample_mon_current - target_charge)
If delta_mon_current > 500mv and V_{BOOST} > 20V then step = 5V
for increase adjustments
(For decrease adjustments a 500mv delta triggers emergency
decrease to minimum Voltage)
If delta_mon_current > 200mv then step = 1V
If delta_mon_current > 100mv and delta_mon_current > 5% *
sample_mon_current then step = 1V

In other examples, new treatments are started with voltage lower than target voltage with a voltage buffer of approximately 10%. The impedance is unknown at the treatment start. These examples save the target_voltage in use at the end of a treatment. If the user has not adjusted the strength parameter manually, it starts a new treatment with saved target_voltage with the 10% buffer. This achieves target current quickly with the 10% buffer to avoid possible over stimulation in case impedance has been reduced. This also compensates for the first 3-4 pulses that are generally higher.

As disclosed, examples apply an initial charge level, and then automatically adjust based on feedback of the amount of current being applied. The charge amount can be varied up or down while being applied. Therefore, rather than setting and then applying a fixed voltage level throughout a treatment cycle, implementations of the invention measure the amount of charge that is being input to the user, and adjust accordingly throughout the treatment to maintain a target charge level that is suitable for the current environment.

The Adaptive Circuit described above provides the means to monitor the charge sent through the electrodes to the user's tissue and to adjust the strength and duration of sending charge so as to adapt to changes in the impedance through the electrode-to-skin interface and through the user's tissue such that the field strength at the target nerve is within the bounds needed to overcome the action potential of that nerve at that location and activate a nerve impulse. These changes in impedance may be caused by environmental changes, such as wetness or dryness of the skin or underlying tissue, or by applied lotion or the like; or by tissue changes, such as skin dryness; or by changes in the device's placement on the user's skin, such as by removing the patch and re-applying it in a different location or orientation relative to the target nerve; or by combinations of the above and other factors.

The combined circuits and circuit controls disclose herein generate a charge that is repeated on subsequent uses. The voltage boost conserves battery power by generating voltage on demand. The result is an effective and compact electronics package suitable for mounting on or in a fabric or similar material for adherence to a dermis that allows electrodes to be placed near selected nerves to be activated.

Fibrous Connective Tissue Healing and Reconstruction

In some example inventions, patch 100, disclosed above, is used for the healing and reconstruction of fibrous connective tissue damage, such as from plantar fasciitis, thus improving the movement of individuals with injuries to such tissues. The present invention stimulates synthesis of collagen in the plantar fasciae, and increases blood flow and angiogenesis in and near fibrous connective tissue, such as plantar fasciitis thereby improving the healing of individuals with injuries to such tissues. The behavior of these individuals is changed to provide better quality movement on a more aggressive schedule, which in turn affects their behavior during daytime activities. Damage to the plantar fasciae from plantar fasciitis is normally noticeable during the first steps taken after awakening from sleep.

Example inventions are directed to improving blood flow as a part of healing in connective tissue such as loose tissue, fibrous tissue, and cartilage. Examples that involve fibrous connective tissue are plantar fasciitis, ankle sprain, Achilles tendon pull, patellar or carpal tunnel syndrome, and “tennis elbow” or lateral epicondylitis. Fibrous connective tissues, such as tendons and ligaments, have less blood flow than muscle tissue and therefore heal more slowly. Limited blood flow in injured fibrous connective tissue lengthens the healing process.

Example inventions provide an integrated system, including patch 100, which may be placed on the skin of the user and activated and used without the help of a medical professional. Examples include hardware and software to selectively stimulate the synthesis of collagen in the plantar fascia as well as stimulate angiogenesis and blood flow in the tissue associated with the injured fibrous connective tissue. The electrical stimulation delivers a targeted application of charge to a specific area in the target tissue.

Collagen fibers are created by fibroblasts in the extracellular matrix. Procollagen molecules assemble together to form collagen fibrils, which then assemble together to form the strong collagen fibers. Type 1 collagen is the most common type in the human body, present in tendons, ligaments, bone, skin and fascia like the plantar fascia.

Electrical stimulation of collagen tissue has been shown to improve the rate of collagen fiber synthesis, especially for Type 1 collagen, which is particular helpful in regenerating damaged tissue. The implementation of the invention eschews implanted stimulation in favor of transcutaneous stimulation of the tissue, avoiding any surgical procedures.

The plantar fascia is a type of deep fascia, or an aponeurosis, which connects the wide muscles of the sole of the foot to the calcaneus, or heel bone. The plantar fascia is only sparingly supplied with blood vessels, causing inflammation or damage to heal slowly. Fasciae are built primarily from collagen and elastin fibers.

Transcutaneous electrical stimulation (“TES”) causes vasodilation to increase blood flow and nutrients for healing. It also causes angiogenesis, which is the creation of new blood vessels, which also increases the blood flow.

Application of the invention to healing of plantar fascia may be extended to other connective tissues in the body, such as Achilles tendon, ankle sprain, tennis elbow, etc.

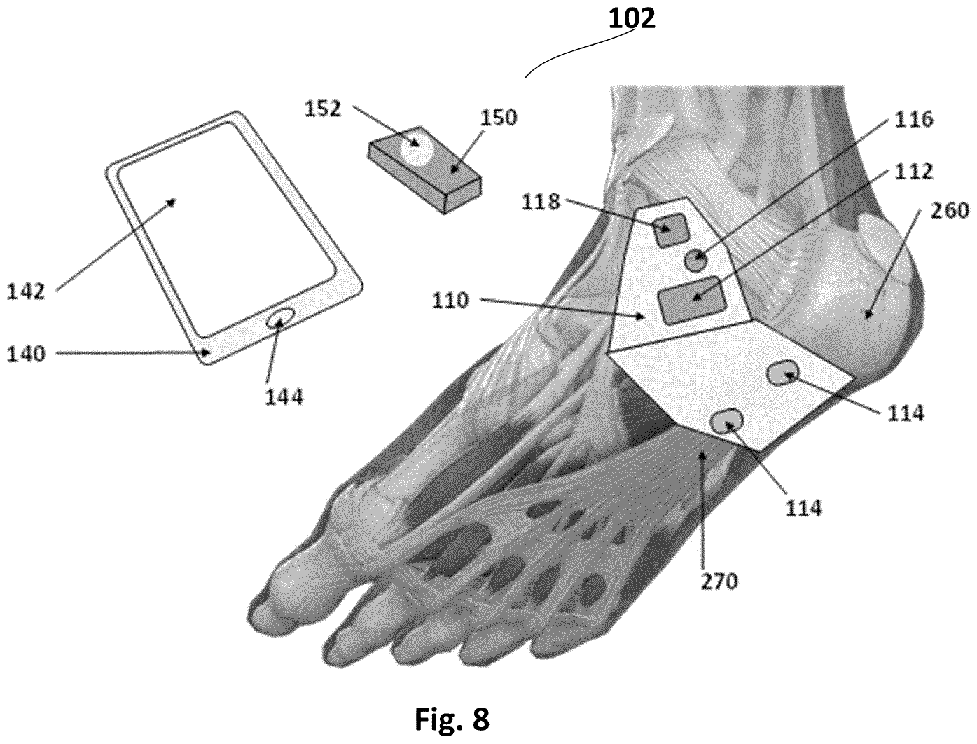

FIG. 8 is an illustration of components of a fibrous connective tissue reconstruction and healing system 102 in accordance with example inventions. System 102, as shown in FIG. 8, is adapted for an injury of the plantar fascia. System 102 includes a healing patch 110, which includes a securing mechanism 112 (e.g., adhesive layer), and one or more electrode pairs 114, with each pair having a positive electrode and a negative electrode (or multiple positive electrodes and a single negative electrode as disclosed below). Patch 110 further includes a power source 116 and a processor 118. System 102 further includes an optional smart controller 140 (e.g., a smart phone), with a display 142, and an acknowledgment button 144, and an optional fob 150 with one or more buttons 152.

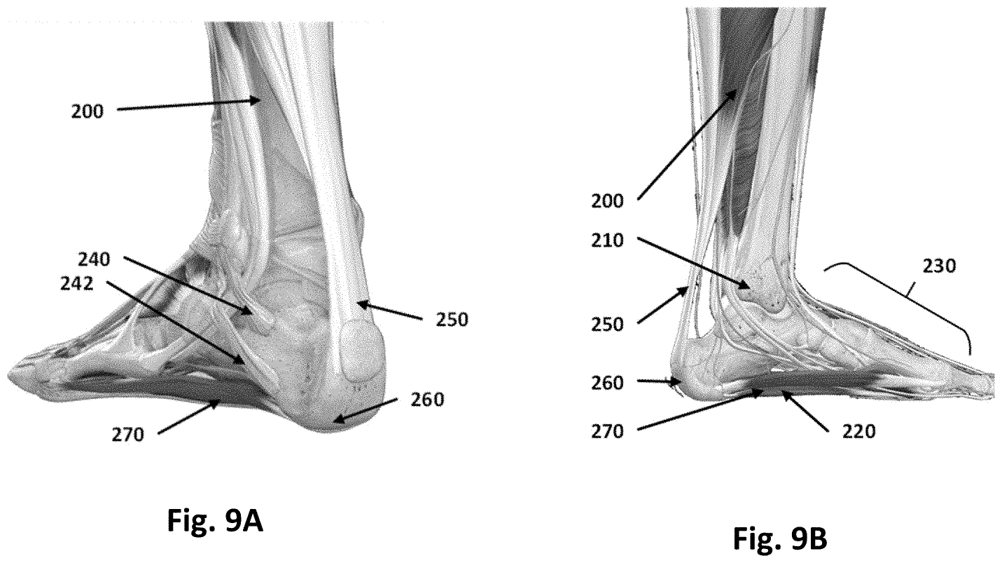

FIG. 9A illustrates the physiology related to plantar fasciitis, including the user 200 right foot with the superior fibular retinaculum 240, the inferior fibular retinaculum 242, the Achilles tendon 250, the calcaneus bone 260, and the plantar fascia 270.

FIG. 9B illustrates the user 200, the ankle 210, the sole 220, the foot 230, the Achilles tendon 250, the medial tubercle of the calcaneus 260, and the plantar fascia 270.

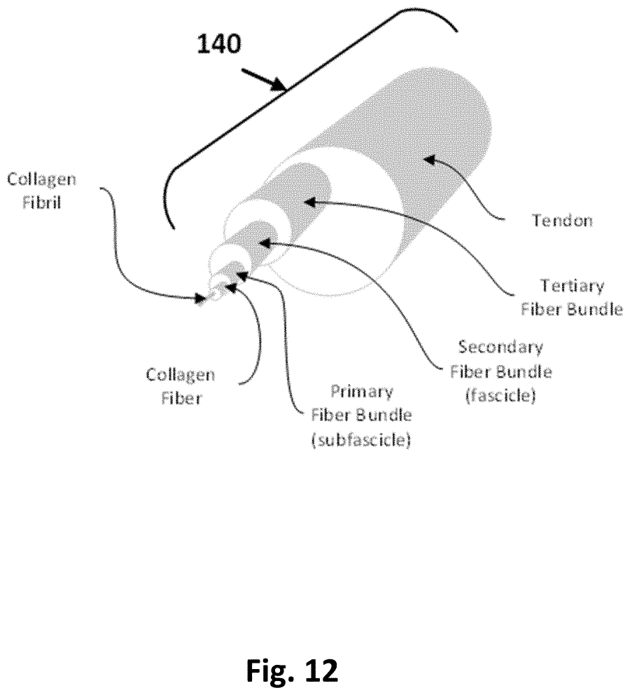

FIG. 12 illustrates the anatomical structure of a tendon. Collagen is formed in the extracellular region. The process depends on secretion of procollagen from cells in the target tissue, such as the plantar fascia, using the exocytosis process. This procollagen, in the form of tropocollagen molecules, assembles in the extracellular space into microfibrils, which then assemble into fibrils, which then assemble into fibers. The fibers are combined with nerve, lymphatic and blood vessels to form bundles called fascicles. The strength of collagen fiber-laden tissue is partly due to the alignment of the collagen fibers. Tissues with higher parallel alignment of collagen fibers is stronger and more resilient. Electrical stimulation has been shown to increase the degree of organization and alignment of collagen fibers during the healing process.

As shown in FIG. 8, healing patch 110 is designed to be placed on the sole of the foot, covering plantar fascia 270. Healing patch 110 is situated so that electrical stimulation may stimulate plantar fascia 270.

Healing patch 110 is designed in a shape to conform to the skin when affixed to the skin and to be electronically effective at stimulating plantar fascia 270. Healing patch 110 is electronically most effective when the positive and negative electrodes are placed axially along the path of the fascia, in contrast to transversely across the path of the fascia which is not as electronically effective.

The shape of healing patch 110 in examples is designed to minimize discomfort for the user 200 when affixed in the target location.

Electrical stimulation has been shown to improve regeneration in collagen, using voltages in the range of 100-500 Volts, pulse widths in the range of 50 to 200 microseconds, pulse repetition rates at both low (2-10 Hz) and high (100 Hz) frequencies, and treatment times of minutes to tens of minutes. Healing patch 110 creates treatment protocols in this same range, adjusted by user 200 for effectiveness and comfort.

The electrical stimulation in accordance with example inventions, improves alignment of collagen fibers in connective tissue during the reconstruction process, which results in more resilient healed connective tissue and less scarring than occurs without the effect of electrical stimulation.

In some examples, healing patch 110 uses one electrode pair 114 to stimulate plantar fascia 270 or other target tissues. In some examples, healing patch 110 uses multiple positive electrodes and one or more negative electrodes to stimulate plantar fascia 270 other target tissues, modifying the waveshapes or timings, or both, of the stimulation pulses from the multiple electrodes to direct the waveform energy at one or more specific points on the fascia other target tissues. Various arrays of electrodes as disclosed above can be controlled to generate optimized stimulation. The stimulation can be adaptive based on feedback from sensors as disclosed above.

In some examples, healing patch 110 uses adhesive surfaces to attach to the skin.

In an example, healing patch 110 fits across the plantar fascia of the right or left foot, including across the calcaneus bone 260, and extending onto the medial or lateral side of the ankle as a continuous substrate, carrying the power source 116, or the processor 118, or both on the portion of the substrate affixed to the ankle, such that the substrate across the plantar fascia fits comfortably during wear by user 200.

In some examples, healing patch 110 (and all other patches disclosed herein) includes one or more sensors which measure internal features or biometrics of the user in the fascia area or other target area. The measurements are used to help the user to orient and place healing patch 110 most accurately in the target location. The sensor data is communicated to one or more of smart controller 140, fob 150 and healing patch 110, and an indication such as LED or vibration is sent to the user to assist them in placing the device.

For example, the orientation vertically or horizontally of healing patch 110 itself can be determined by a 9-axis accelerometer on the patch. A smart phone app executed on smart controller 140 can tell the user in real-time to rotate the patch to the proper orientation before sticking it to the skin. The shape of healing patch 110 can be designed in a shape to assist the user in orienting it properly. Further, a marking (e.g., an arrow meant to be vertical) could be printed on the patch or on a removable paper liner (so that the arrow is removed when the patch is actually applied).

Further, healing patch 110 can be designed to accommodate multiple orientations. For example, the electrodes could be an array or series or matrix of sub-electrodes, and the patch could select which to use for effective stimulation based on the position and orientation of the patch. Similarly, healing patch 110 can include two microphones which could have their roles reversed if the patch were placed “upside down” on the skin.

Further, the position of healing patch 110 on the foot (or any other location in examples) could be deduced after the patch is affixed to the skin by sensing through the skin with the on-board sensors, then notifying the user through the app that the patch is good or that it needs to be re-positioned.

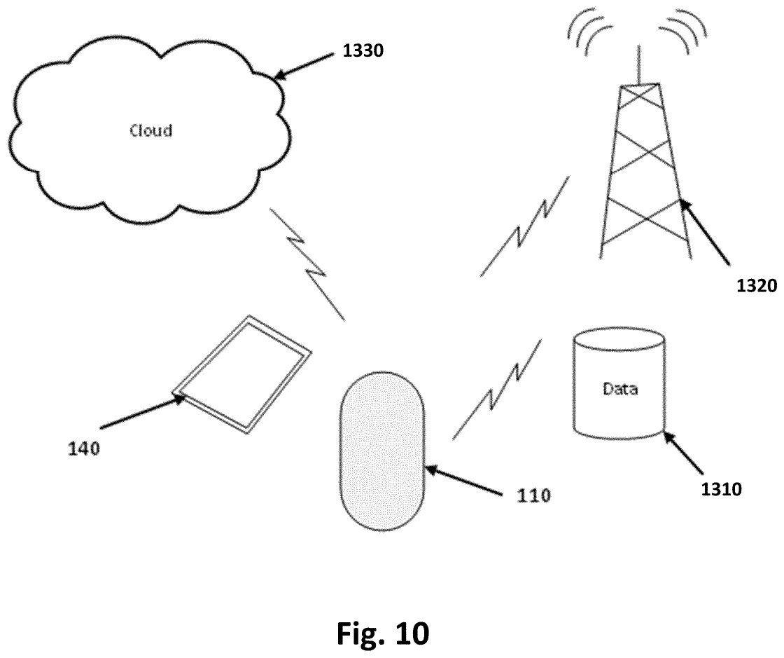

FIG. 10 illustrates the connectivity of patch 110 and smart controller 140 with a data store 1310, a network 1320, and the cloud 1330 in example inventions.

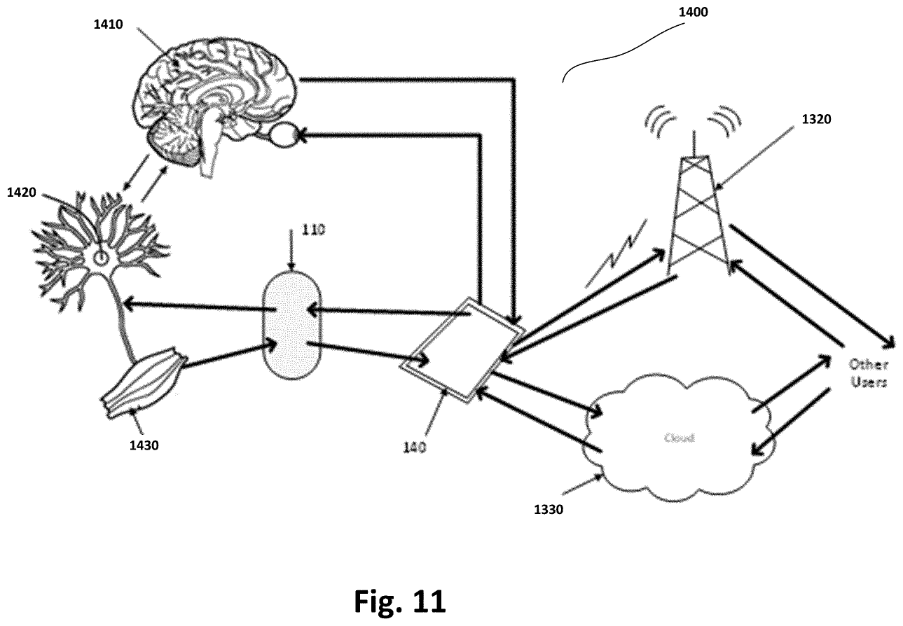

FIG. 11 illustrates a feedback loop to create a closed-loop system between user 200, patch 110 and smart controller 140 in example inventions.

The sensors of patch 110 may be of several different modalities including pressure sensors, temperature, humidity (sweat), Electromyography (“EMG”) sensors, motion, and accelerometers. The sensors can gather biometric data about the user 200 such as the number of steps taken, gait information, contact sequencing of various parts of the foot with the ground, and environmental conditions, such as road surface. The data is gathered by smart controller 140 and/or fob 150, and sent to data store 1310, the network 1320 (e.g., the Internet), or directly to the cloud 1330 via the wireless connection.

In examples, variations of patch 100 are designed to stimulate other fibrous connective tissues, such as aligament or a tendon, where the shape of the patch is designed to conform to the location of stimulation, and the stimulation protocol is designed to stimulate that specific tissue, such as in a knee, shoulder, elbow, ankle or any joint that includes fibrous connective tissue.

In one example, fibrous connective tissue healing system 102 stimulates repair to damage to tissue in the superior fibular retinaculum 240, the inferior fibular retinaculum 242, or the Achilles tendon 250.

Healing patch 110, smart controller 140, and fob 150 may be combined in a variety of ways to implement the fibrous connective tissue healing system 102. In some examples, user 200 uses fob 150 to send data and controls to smart controller 140. In some examples, user 200 uses fob 150 to send data and controls to patch 110.

In some examples, user 200 uses smart controller 140 directly, and a fob 150 is not used.

In some examples, fob 150 communicates data and controls with smart controller 140 or to patch 110, or both, through wireless means, through the use of Bluetooth Low Energy (“BLE”), Wi-Fi, or other means.

In some examples, power source 116, smart controller 140, and fob 150 may be powered by battery or rechargeable means.

In some examples, patch 110 sends an activation signal to the relevant tissue and repeats this signal according to a timer preset by the user 200, where the interval between stimulations being selected to effectively optimize the synthesis of collagen. In some examples, analysis of measurements from smart controller 140 may be performed by processing in a remote server, in the cloud, or on a computer separate from smart controller 140 but local to the user, such as a personal computer.

Referring again to FIG. 11, in this example, the patch 110 (e.g., as patch 100 or patch 120) is capable of applying stimuli to fibrous connective tissue, such as Achilles tendon 250 or the plantar fascia 270, or other organs such as the brain 1410 through afferent peripheral pathways. These actions may be sensed by patch 100, which may act on the information to modify the stimulation it provides. This closed loop constitutes the first level of the system 1400 in this example.

Patch 110 may also be caused to operate by signals received from a smart controller 140, such as a cellphone, laptop, key fob, tablet, or other handheld device and may transmit information that it senses back to smart controller 140. This constitutes the second level of system 1400 in this example.

Smart controller 140 is caused to operate by commands from a user 200, who also receives information from smart controller 140. The user may also receive information about actions of the body via natural senses such as vision or touch via sensory nerves and the spinal cord, and may in some cases cause actions in the body via natural pathways through the spinal cord to the connective tissues.

Smart controller 140 may also communicate information to other users, experts, or application programs via network 1320 or via the cloud 1330, and receive information from them via network 1320 or via the cloud 1330.

The user 200 may choose to initiate or modify these processes, sometimes using protocol applications residing in patch 110, smart controller 140, or the network 320, such as the Internet or wireless networks. This software may assist the user, for example, by processing the stimulation to be delivered to the body to render it more selective or effective for the user, and/or by processing and displaying data received from the body or from network 1320 to make it more intelligible or useful to the user.

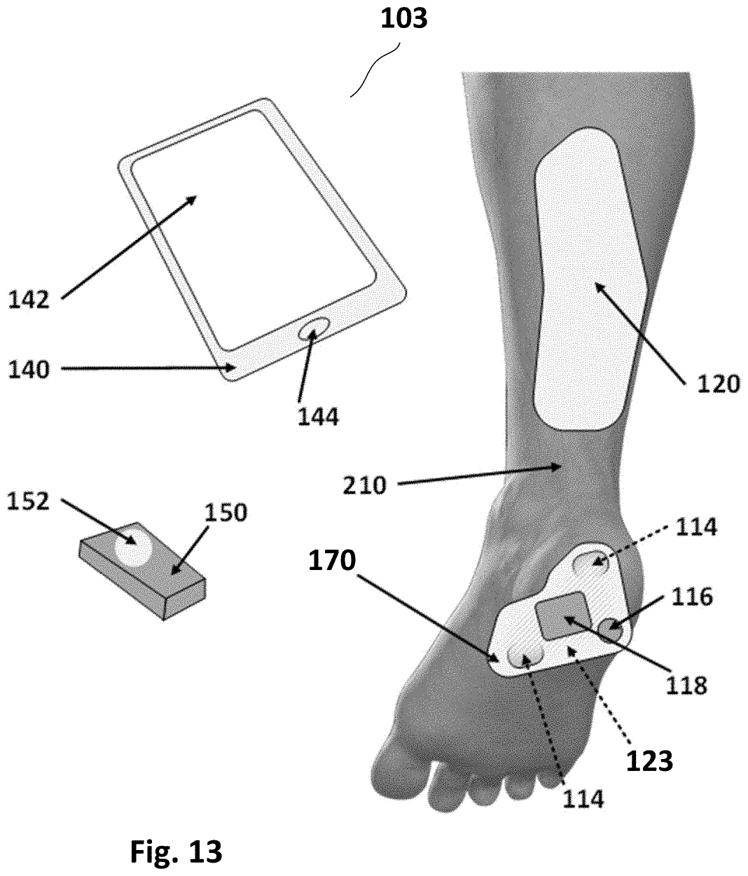

FIG. 13 is an illustration of components of a fibrous connective tissue reconstruction and healing system 103 in accordance with example inventions. System 103, similar to system 102 of FIG. 8, is adapted for an injury of the plantar fascia. System 103 includes a healing patch 170, which includes a substrate 123, and one or more electrode pairs 114, with each pair having a positive electrode and a negative electrode (or multiple positive electrodes and a single negative electrode as disclosed below). Patch 170 further includes a power source 116 and a processor 118. System 103 further includes an optional smart controller 140 (e.g., a smart phone), with a display 142, and an acknowledgment button 144, and an optional fob 150 with one or more buttons 152. System 103 further includes an optional Achilles patch 120.

Healing patch 170 applies electrical stimulation to the surface of the skin with its two or more electrode pairs 114, transcutaneously stimulating the underlying target tissues. Smart controller 140 and fob 150, separately or together, schedule the stimulations including the detailed protocol of signal strength, timing and duration of treatment. The treatment may be started and stopped according to a schedule managed by smart controller 140 or fob 150, or both, or one or both of the start and stop may be triggered by actions of user 200 on the buttons 152 or the display 142. The user applies patch 170 at a target location such that the electrical stimulation is effective at the target tissue.

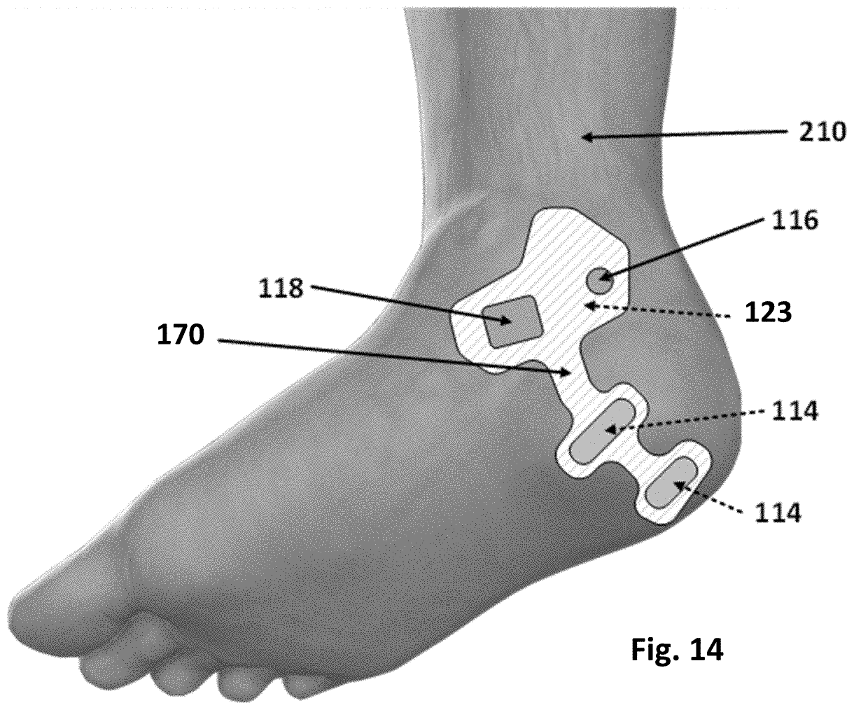

FIG. 13 shows how healing patch 170 is designed to be placed on the heel and sole of the foot so that electrical stimulation may increase blood flow to the plantar fascia 270 using electrical fields. The shape of healing patch 170 is designed to conform to the skin when affixed to the skin to be electronically effective at stimulating blood flow and angiogenesis. FIG. 14 illustrates healing patch 170 conforming to the shape of the angle and sole of the foot in accordance with example inventions.

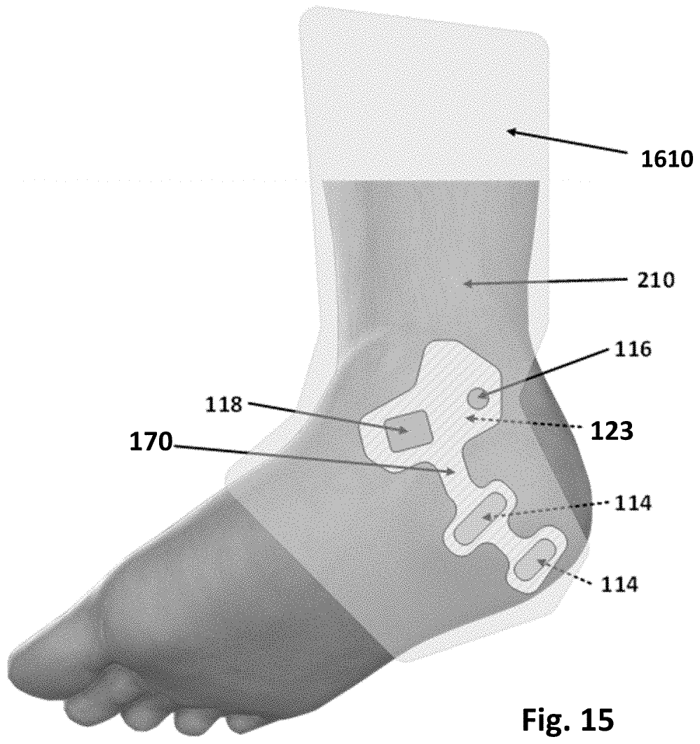

FIG. 15 illustrates healing patch 170 affixed to the interior side of a bandage 1610 in accordance with example inventions. As shown in FIG. 15, healing patch 170 is affixed inside a bandage 1610 and the bandage is mounted to the skin with sufficient force or tension to hold healing patch 170 in place on the skin without the use of adhesive on the skin side of healing patch 170.

In some examples, healing patch 170 connects the elements of its one or more electrode pair 114 by extending wired connections to those elements from power source 116 through a contiguous and extended substrate 112, lengthened to allow the electrode elements to reach optimum locations over the target tissues, and positioning the power source and the processor to optimize comfort, as shown by example in FIG. 15.

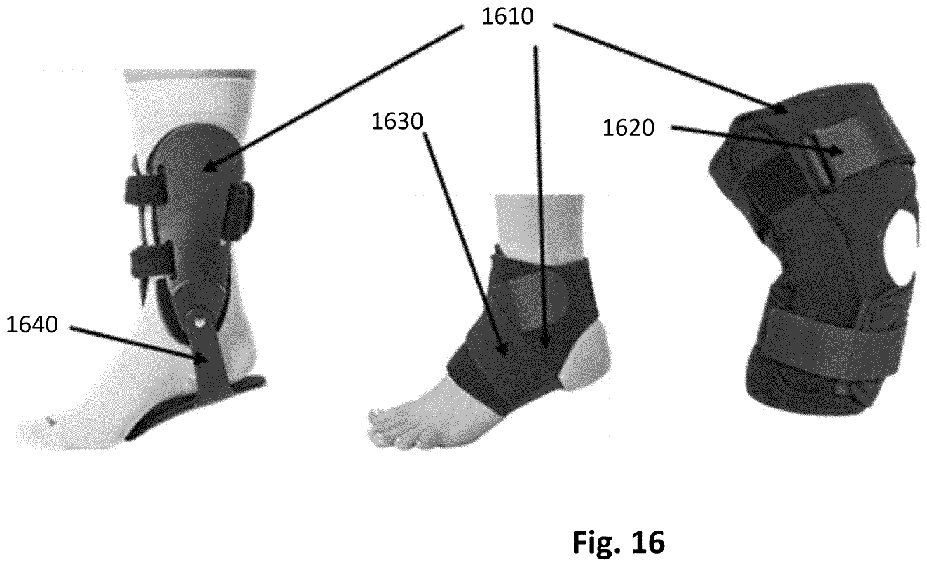

FIG. 16 illustrates several bandage arrangements with the patch in accordance with example inventions. Bandage 1610 shown in FIG. 16 are designed to hold patch 170 in the target location on the skin. Bandage 1610 in example inventions is a device or system with dimensions designed to conformally fit in the target area and to stay in place using adhering aspects such as adhesives, closures 1620, hook-and-loop fasteners 1630, etc. Bandage 1610 may incorporate a support brace 1640. Examples of bandage 1610 may wrap around the target location, or may open into a flat device to be affixed around the target location with straps or tabs, or may be a cylinder to be pulled around the limb or other target location and held in place by elastic materials.

In some examples, two or more patches 170 are affixed to the inside of bandage 1610 to perform a stimulation on coordination by one or more of smart controllers 140 or fobs 150 or both.

In some examples, bandage 1610 opens before application to the ankle foot to allow affixing patch 170 to the inside surface of the bandage. In some examples patch 170 is affixed to the inside surface of bandage 1610 through an access port or flap.

In some examples the user inserts patch 170 inside bandage 1610 by reaching inside bandage 1610 before applying bandage 1610 to the ankle foot 230. In some examples, patch 170 is affixed to the inside surface of bandage 1610 using adhesive. In some examples, patch 170 is affixed to the inside surface of the bandage 1610 using tabs or fasteners. In some examples, patch 170 is built into bandage 1610 as part of the bandage production process.

In some examples, patch 170 is affixed to bandage 1610 so that the patch 170 is used for one application to the skin, patch 170 then discarded after the application to the skin by removing bandage 1610 from the skin and removing patch 170 from bandage 1610.

In some examples, patch 170 is affixed to the bandage 1610 so that patch 170 is used for multiple applications to the skin, bandage 1610 removed from the skin between sessions without removing patch 170 from bandage 1610, repeating until smart controller 140 or fob 150, or both, indicate to the user 200 that patch 170 must be removed, discarded and replaced with another patch 170.

In some examples, patch 170 is designed to stimulate the skin for one session with a duration and intensity set by smart controller 140 or fob 150, or both, patch 170 then removed from the skin after the stimulation is completed.

In some examples, patch 170 is designed to stimulate the skin for multiple sessions, the duration, intensity and delay between sessions set by smart controller 140 or fob 150, or both, patch 170 remaining on the skin between sessions and removed after the last session for patch 170.

In some examples, patch 170 is designed to stimulate the skin for multiple sessions, the duration, intensity and delay between sessions set by smart controller 140 or fob 150, or both, patch 170 inside the Bandage 610 with the Bandage remaining on the skin between sessions and removed after the last session for that patch 170.

Electrode Arrangements

In examples, patch 100 (including any other patches disclosed herein) can use multiple positive electrodes in an array or matrix and also include multiple negative electrodes. Each positive electrode creates an electric field with the negative electrode nearest to it, such that the charge flows from one electrode to the other. Each positive electrode's field is not affected by other negative or positive electrodes, as these other electrodes are electrically distant from the positive electrode and the negative electrode. However, this set of electrodes may complicate the physical and electrical layout of the patch.

Therefore, in example inventions, a set of positive electrodes instead shares only one common negative electrode, such that the return current path back to the stimulating circuit is through the one negative electrode. This common negative electrode is larger than individual negative electrodes for each positive electrode when considering the two approaches on a fixed patch area. By making the common negative electrode larger, its impedance can be lower to the skin, its fringe area is minimized such that uncomfortable stimulation sensations are minimized when compared to current paths through small electrodes, and leakage currents are minimized because the single, larger negative electrode may be more easily isolated from circuitry than a multiplicity of negative electrodes.

The set of positive electrodes may be positioned in various ways, such as around the perimeter of the patch, to provide effective stimulation when the patch is placed over the damaged area with a range of accuracy. For example, a patch may be placed over a damaged area, the patch may be able to more effectively stimulate one area of the damaged area at a time due to the power limitations of the patch. The power limitation may be due to the battery selected, or due to the maximum driving current from the stimulation circuit, or other factors. With a set of positive electrodes each returning current through a common negative electrode, the patch is able to create therapeutic electric fields over one part of a wound for a period of time, and then move to a new part of the damaged area for a second period of time, and so on until the entire damage is treated. This sequence of treatment periods may be executed without removing or repositioning the patch. The sequence may require replacement of the power supply, such as a new battery or recharging a rechargeable battery with non-contact charger, but the controller remembers the last-treated section of the damaged area and resumes with the next treatment area when it has refreshed power source.

In one example, the sensor on the patch detects the progress of healing of each region of the damaged area under the patch. As each region is healed, the controller reduces the use of the positive-to-negative electrode stimulating path over that region of the damaged area.

An example, the sensor on the patch detects that a previously-healed portion of the damaged area has reopened, such as through user's movement. The controller monitors the state of each region of the wound and adjusts the application of stimulation treatment from one positive electrode to another, thereby maintaining effective healing over the entire area of the damaged area. Stimulation in an area is stopped when the patch detects a healed area, and stimulation may resume if an area is sensed as reopened or re-injured.

An example, the patch may lack sufficient means of energy delivery to effectively heal the entire damaged area at one time over a period of treatment. In such a case, the patch may be placed over the damaged area and left in place for the duration of treatment, as the patch stimulates one region at a time according to the limit of its rate of energy delivery.

The set of positive electrodes may be connected to the stimulating circuit one at a time or more than one at a time, using low-impedance switches between the shared voltage generating stimulation circuit and the individual electrodes. The switches are controlled by the controller, such that only the desired positive electrode or electrodes are connected at one time.

The patch may use one positive electrode and a set of negative electrodes. The positive electrode is driven by the voltage for stimulation, using one circuit and working through the lower impedance of the large, common positive electrode in its contact with the skin. The negative electrodes may be a common ground, and connected to each other by conductive paths on the patch and further back to the stimulating circuit to complete the current loop. Alternatively, each negative electrode may be connected to the common ground through a low-impedance switch, the switches being under control of the controller, such that only the desired negative electrode or electrodes are connected to ground at one time, thereby limiting the return current path.

The set of positive electrodes driven by a stimulation voltage may have individually adjusted stimulation voltages such that, when connected and stimulating the skin, the combined stimulation from multiple positive electrodes is more effective than identical stimulation waveforms from all positive electrodes. The currents from each of the positive electrodes passes through the common negative electrode and back to the stimulating circuit. Individual stimulating waveforms are created by individual stimulating circuits which have specific setups under control of the controller. The controller may adjust the amplitude, phase, pulse width, and frequency of each circuit to create a combination of stimulation through multiple positive electrodes.

In general, when patch 100 is applied to the skin and then uses sensors to detect when to stimulate, it uses sensing circuits that are separate from the circuits used for electrical stimulation. When the detection mechanism involves electrical signal sensing, the sensors use electrodes on the skin-facing surface of the patch. The controller monitors certain conditions through electrical signal sensing, then turns electrical stimulation on or off according to the treatment regime associated with the sensed condition. For example, muscle twitching may be detected by electromyography (“EMG”). Patches use separate sensing electrodes and stimulation electrodes since each as different requirements.

However, separate sensing and stimulating electrodes increases the size of the patch and may require accurate placement of the patch. In contrast, in some examples, patch 100 uses the same set of electrodes for sensing as for stimulating. The connections to the controller are shared between sensing and stimulating functions, or the connection to each electrode is routed to unique controller pins with a low-impedance switch. The state of the switch is controlled by the controller, multiplexing sensing and stimulating functions.

Sensing requires a relatively high-impedance path from the skin surface to the analog-to-digital converter (“ADC”) circuit. The ADC may be a discrete component, passing a digital signal on to the controller, or the ADC may be integrated in the controller on one or more pins. High-impedance is required to generate a voltage proportional to the biometric, such as in EMG, the voltage having a range large enough to discriminate a wide set of values when digitized.

Stimulation requires a relatively low-impedance path to the skin surface, such that the driving circuit can overcome the impedance and drive energy into the tissue for treatment.

The two competing requirements may be combined through the use of a low-impedance or matched-impedance switch. The switch routes the signal captured at the electrode to either the sensing pin or the driving pin. For example, a single pin on the controller may be programmable to low- or high-impedance, and be able to both sense and drive into its load.

In another example, a small part of a larger stimulating electrode may be electrically isolated in the layout such that the small part may work as a sensing electrode when connected to the sensing circuit, and yet may work as part of the overall stimulating electrode when connected to the stimulating circuit. The isolation may be through two switches, one with low impedance for the sensing function, the other with impedance matching the overall impedance of the larger electrode. This latter aspect helps to minimize reflections and aberrations in the stimulating waveform when the stimulating circuit drives both the larger electrode area and the connected smaller area.

In another example, a patch uses a set of small electrodes to stimulate the skin. The overall impedance of the stimulating patches in combination is low, to optimize the effectiveness of the stimulation. The impedance of each individual small electrode is higher, such that it is effectively used in a sensing circuit.

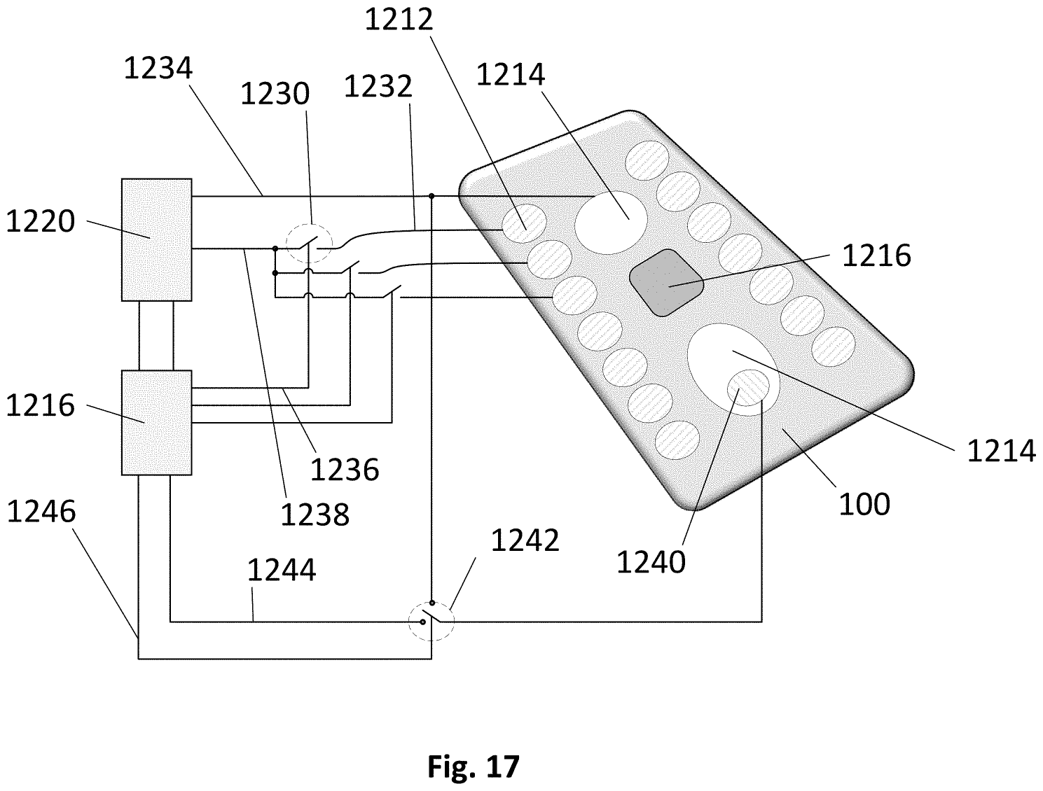

FIG. 17 illustrates patch 100 with multiple electrodes that are adapted to provide both stimulation and sensing in accordance with example inventions. Patch 100 includes a set of 14 positive electrodes 1212; and a set of 2 negative electrodes 1214. Patch 100 further includes a processor 1216 shown in a physical view and schematic view. Patch 100 further includes a stimulation voltage circuit 1220, a set of stimulation switches 1230 with a stimulation voltage wire 1232 and a return current wire 1234. Patch further includes a stimulation switch control wire 1236, and a sensor electrode 1240 with a sensing wire 1244, a sensing mode switch 1242, and a sensing mode wire 1246. FIG. 17 illustrates only 3 of the necessary 14 stimulation switches and associated wires that would be included in this example invention.

In operation, patch 100 selects one or more of positive electrodes 1212, connecting each to stimulation voltage circuit 1220 with the corresponding stimulation switch 1230. The stimulation voltage passes from stimulation voltage circuit 1220 to all of the selected positive electrodes 1212, then as a field to negative electrodes 1214, and back to stimulation voltage circuit 1220. In example inventions, patch 100 selects the subset of the available positive electrodes 1212 to optimize the stimulation of the underlying tissue. The selection is adjusted in the software or firmware of processor 1216 according to the positioning of patch 100 on or near the target area.

Further, in example inventions, patch 100 selects the one or more sensor electrodes 1240 by activating sensing mode switch 1242 to connect the sensor to processor 1216. Processor 1216 uses one or more of hardware or software or firmware to analyze the measurement procured from sensor electrode 1240, using the analyzed measurement to inform the selection of positive electrodes 1212. Patch 100 changes the mode of sensing mode switch 1242 to connect sensor electrode 1240, or to return current wire 1234 when the electrode is used during a stimulation.

Audio Input

Users who apply patch 100 to their skin to heal may be required by the patch to also use a separate controller (i.e., smart controller 140), such as a smart phone, to activate the patch and continue to control the treatment. Some users are resistant to using a smart phone because they do not own one and do not wish to purchase one, or are hesitant to deal with the complexities of smart phone usage. Such users will be unable to manage the use of a patch applied to their skin if that patch requires the use of a smart device.

Therefore, in example inventions, patch 100 is a “self-sufficient” patch that frees the user from using a smart phone. This is particularly important for the population which is still resistant to using a smart phone, or unable to use a smart phone due to other medical condition limitations.

In example inventions, patch 100 includes an audio sensor which can detect a audio input such as nearby speech and pass that audio stream to the on-patch controller for analysis. The on-patch controller can, through voice analysis, detect the use of key words or phrases that can be understood to start, stop or otherwise control the patch's stimulation protocol.

A subset of the set of functions provided by a smart phone when connected wirelessly to a patch may be provided using voice control through the audio sensor on the patch.

The patch may include a multi-axis accelerometer which can detect the user's position, such as standing or lying prone. The controller can then apply the healing treatment only at prescribed times correlated to the user's position. For example, the treatment may be applied only when the user is lying prone or supine. For example, in treating plantar fasciitis which creates a painful sensation when the user first moves from sitting or lying down to a standing or walking position, the controller may apply the healing treatment for a prescribed number of minutes when its accelerometer first detects movement.

In an example, the patch may apply healing treatment only during such times as when the healing treatment is most effective, such as during sleep. Side effects of the healing which may be distracting during waking hours may not affect sleep. Therefore, the healing treatment may be amplified during sleep, the sleep periods being determined on the patch by the accelerometer.

An example, the patch may start and stop healing treatment whenever the user changes position from a treatment-appropriate position to a treatment-inappropriate position. For example, treating during sleep may be effective, but treatment is stopped whenever the user gets out of bed to use the bathroom. Such on-and-off control optimizes power usage.

Data Manager

In examples, patch 100 includes a data manager implemented by control unit/processor 1002, that has primary responsibility for the storage and movement of data to and from the communications controller, sensors, actuators, and a master control program. The data manager has the capability to analyze and correlate any of the data under its control. It provides logic to select and activate nerves. Examples of such operations upon the data include: statistical analysis and trend identification; machine learning algorithms; signature analysis and pattern recognition, correlations among the data within a data warehouse, a therapy library, tissue models, electrode placement models, and other operations. There are several components to the data that is under its control as disclosed below.

The data warehouse is where incoming data is stored; examples of this data can be real-time measurements from the sensors, data streams from the Internet, or control and instructional data from various sources. The data manager will analyze data that is held in the data warehouse and cause actions, including the export of data, under master control program control. Certain decision making processes implemented by the data manager will identify data patterns both in time, frequency, and spatial domains and store them as signatures for reference by other programs. Techniques such as EMG, or multi-electrode EMG, gather a large amount of data that is the sum of hundreds to thousands of individual motor units and the typical procedure is to perform complex decomposition analysis on the total signal to attempt to tease out individual motor units and their behavior. The data manager will perform big data analysis over the total signal and recognize patterns that relate to specific actions or even individual nerves or motor units. This analysis can be performed over data gathered in time from an individual, or over a population of patch users.

The therapy library contains various control regimens for patch 100. Regimens specify the parameters and patterns of pulses to be applied by patch 100. The width and amplitude of individual pulses may be specified to stimulate nerve axons of a particular size selectively without stimulating nerve axons of other sizes. The frequency of pulses applied may be specified to modulate some reflexes selectively without modulating other reflexes. There are preset regimens that may be loaded from the cloud or 3rd party apps. The regimens may be static read-only as well as adaptive with read-write capabilities so they can be modified in real-time responding to control signals or feedback signals or software updates. One such example of a regimen has parameters A=40 volts, t=500 microseconds, T=1 Millisecond, n=100 pulses per group, and f=20 per second. Other examples of regimens will vary the parameters within ranges previously specified.

The tissue models are specific to the electrical properties of particular body locations where patch 100 may be placed. Electric fields for production of action potentials will be affected by the different electrical properties of the various tissues that they encounter. The tissue models are combined with regimens from the therapy library and the electrode placement models to produce desired actions. Tissue models may be developed by MRI, Ultrasound or other imaging or measurement of tissue of a body or particular part of a body. This may be accomplished for a particular user and/or based upon a body norm. One such example of a desired action is the use of a tissue model together with a particular electrode placement model to determine how to focus the electric field from electrodes on the surface of the body on a specific deep location corresponding to the nerve in order to stimulate the nerve selectively to reduce incontinence of urine, or to promote healing of damaged tissue. Other examples of desired actions may occur when a tissue model in combination with regimens from the therapy library and electrode placement models produce an electric field that stimulates a tibial nerve. Many other examples of desired actions follow for the stimulation of other nerves.

Electrode placement models specify electrode configurations that patch 100 may apply and activate in particular locations of the body. For example, patch 100 may have multiple electrodes and the electrode placement model specifies where these electrodes should be placed on the body and which of these electrodes should be active in order to stimulate a specific structure selectively without stimulating other structures, or to focus an electric field on a deep structure. An example of an electrode configuration is a 4 by 4 set of electrodes within a larger array of multiple electrodes, such as an 8 by 8 array. This 4 by 4 set of electrodes may be specified anywhere within the larger array such as the upper right corner of the 8 by 8 array. Other examples of electrode configurations may be circular electrodes that may even include concentric circular electrodes. Patch 100 may contain a wide range of multiple electrodes of which the electrode placement models will specify which subset will be activated. Electrode placement models complement the regimens in the therapy library and the tissue models and are used together with these other data components to control the electric fields and their interactions with nerves, muscles, tissues and other organs. Other examples may include patch 100 having merely one or two electrodes, such as but not limited to those utilizing a closed circuit.

Stack-Up of the Patch

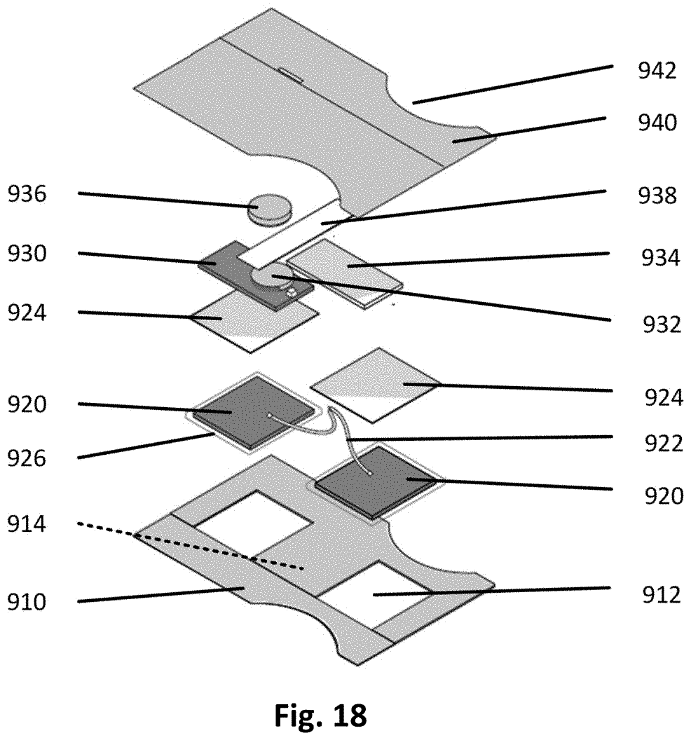

FIG. 18 illustrates a stack-up view of patch 100 in accordance with example inventions. A bottom layer 910 is a fabric tape with adhesive on the skin-facing side. A hole 912 is cut into the bottom layer for each of the electrodes 920. A removable paper 914 adheres to the adhesive on the skin-facing side of bottom layer 910. Two or more electrodes 920 are coupled by a wire 922 to a printed circuit board assembly (“PCBA”) 930.

Electrodes 920 are covered with a polyimide tape A 924 to prevent short circuits from electrodes 920 to PCBA 930 and to prevent movement of electrodes 930 within the layers of the assembly. Each electrode 930 is coated on the skin-facing surface with hydrogel 926. Each electrode 920 has a release layer covering hydrogel 926. A battery clip 932 is attached to PCBA 930. A battery 936 is inserted into battery clip 932. A battery pull tab 938 is inserted into battery clip 932. PCBA 930 is wrapped in polyimide tape B 934 to restrict access by the user to the electronics. A top layer 940 of fabric tape with adhesive on the PCBA-facing side is stacked on top to complete the assembly. Ankle bone cutouts 942 are designed into the shapes of bottom layer 910 and top layer 940 to accommodate the ankle bone and to assist the user to correctly place patch 100.

Hydrogel Adaptation

Variations in the viscosity and composition of hydrogel 926 leads to variation in the migration of the substance from its original area on each electrode to a wider area, possibly touching the skin outside the dimensions of patch 100. As the hydrogel migrates, its electrical performance changes. The circuitry on PCBA 930 measures the voltage applied to the skin in real-time during the course of each treatment. The adaptive circuit calculates the charge delivered to the skin, which is a function of many parameters, including the conductivity of hydrogel 926. Therefore, the performance of patch 100 is maintained while the hydrogel portion of the device changes its performance. The adaptive circuit adjusts the delivery of charge to also account for all changes in body and skin conductivity, perspiration and patch contact.

As the performance of the hydrogel 926 decreases with time, the adaptive circuit and the firmware in PCBA 930 records the expected life of the specific patch while it is powered on and on the skin of the user. When patch 100 determines that the device's lifetime is near an end, the firmware signals to the fob or smart controller, such that the user receives an indication that this patch has reached its limit.

Crimped Connection from Electrode to PCBA

Each electrode 920 is coated with hydrogel 926 when the electrode is manufactured. In some examples, a wire 922 is connected to both the electrode and the PCBA 930 in a permanent fashion, such as by soldering, when electrodes 920 are manufactured. The electrode-plus-wire-plus-PCBA assemblies are each enclosed in an airtight bag until they are subsequently assembled with the tapes and adhesive layers to form a complete patch 100. Due to the complex nature of these assembly steps, the hydrogel on the electrodes may be exposed to air and humidity for a period of time which affects the life expectancy of the hydrogel.

In an example, electrodes 920 are coated with hydrogel 926 but no wire is attached at that stage. Instead, a small clip is soldered to each electrode which does not affect the hydrogel nor attach the electrode to any larger assembly which would require longer time in the assembly line. These coated electrodes are each encased in an airtight bag with a heat seal or other means. The hydrogel does not degrade during the time that the coated electrode is inside the sealed bag.

In an example, wire 922 is inserted into the small clip which had previously been soldered to electrode 920, this connection being stronger and less prone to defect than the soldering or attachment of the wire strands directly to electrode 920. The clip and the wire do not affect hydrogel 926. Each coated electrode 920, with its clip and attached wire, is encased in an airtight bag with a heat seal or other means. Hydrogel 926 does not degrade during the time that the coated electrode is inside the sealed bag. The coated electrodes 920 are removed from their airtight bags only immediately before they are connected to PCBA 930.

An additional benefit of separating the coated electrodes 920 from PCBA 930 as two different subassemblies until put into a completed patch 100 is that coated electrodes found to be defective or expired from too lengthy time on the shelf may be discarded without the expense of discarding an already-attached PCBA. The more expensive PCBAs have a shelf life independent of the shelf life of the coated electrodes. These two subassemblies' inventories may be stocked, inspected and managed independently. This reduces the overall cost of manufacture of patches 100 devices without affecting their performance.

Die Cut Fabric Tape

In some examples, bottom layer 910 is placed as a layer over electrodes 920 using a solid layer of fabric tape. The overall thickness of patch 100 is therefore partly determined by the thickness of the fabric tape over electrodes 920. Further, in order to place electrodes 920 on the layer of fabric tape securely, the paper cover on the fabric tape must be pulled back to expose the adhesive coating. This results in a degradation of the adhesive properties of the tape.

In examples of patch 100, bottom layer 910 fabric tape is cut to create holes 912 for each of electrodes 920, according to the defined sizes of those components. Each electrode 920 is placed in the corresponding hole, without the added thickness of a fabric tape layer on top. Since no paper cover needs to be pulled back to mount electrodes 920 to the fabric tape, the adhesive of the fabric tape is not affected. The holes may be cut with a die in order to create accurate edges, without tears or fibers which may interfere with electrodes 920.

Contoured to Ankle Bone

In some examples, patch 100 has a rectangular shape. This allows PCBA 930, battery 936 and electrodes 920 to fit in between fabric and adhesive bottom layer 910 and top layer 940, and to be affixed to the skin by the user, then to be peeled away and discarded after use. In some examples, patch 100 has a shape contoured to the position in which it is to be affixed to the skin. The reference point in properly positioning patch 100 is the malleolus, or ankle bone in some example uses. Therefore, patch 100 has an ankle bone cutout 942 along the vertical side, this cutout accommodating the ankle bone when patch 100 is placed close alongside the ankle bone.

In some examples, cutout 942 is designed into patch 100 on only one side, such that battery 936, PCBA 930 and electrodes 920 are properly aligned on one of the left or the right ankle. Patch 100 can then be offered in two varieties—one for the left ankle with cutout 942 on the first vertical side, and one for the right ankle with cutout 942 on the second vertical side.

In some examples, cutout 942 is designed into patch 100 on both vertical sides, such that battery 936, PCBA 930 and electrodes 920 are properly aligned on either of the left or right ankle. Patch 100 can then be offered in only one variety.

Battery and Battery Tab

Patch 100 includes battery 936, which is enclosed by battery clip 932, assembled onto PCBA 930. During manufacturing, battery 936 is inserted into battery clip 932 to secure it from dropping out. In addition to the battery itself, battery pull tab 938 is placed between one contact of battery 936 and the corresponding contact in battery clip 932. Battery pull tab 938 prevents electrical connection between battery 936 and battery clip 932 at that contact until battery pull tab 938 is removed. When in place, there is an open circuit such that patch 100 is not activated and does not consume power until battery pull tab 938 is removed.

In some examples, battery pull tab 938 is designed to be removed by pulling it out in the direction opposite that in which battery 936 was inserted into battery clip 932. This pulling action may lead to movement of the battery itself since it experiences a pulling force toward the open side of battery clip 932. This battery movement may cause patch 100 to cease operating or to never activate.

In one example, battery pull tab 938 and battery clip 932 are designed so that battery pull tab 938 is pulled out in the same direction as battery 936 was pushed into battery clip 932. Therefore, the force pulling battery pull tab 938 out of patch 100 serves only to make battery 936 more secure in its battery clip 932. This reduces the chance of inadvertent movement of battery 936 and the effect on activation or operation of patch 100.

Electrode Release Film

Each of electrodes 920 in the assembled patch 100 is covered with a Polyethylene Terephthalate (“PET”) silicon covered release film 926. The release film is pulled away by the user when patch 100 is affixed to the skin. In some examples, the PET silicon covered release film 926 is transparent. This may lead to instances of confusion on the part of the user when the user may not be able to determine if the tape has been removed or not. Affixing patch 100 to the skin with any of electrodes 920 still covered with tape will cause patch 100 to be ineffective. This ineffectiveness may not be noticed until the first treatment with patch 100. If the affixed patch 100 is found to be ineffective when the user is feeling an urge to urinate, the user may struggle to either properly void their bladder or to remove patch 100, peel off the tapes from the electrodes or affix a new patch 100 and suppress the urge with the re-affixed or new device.

In examples, PET silicon covered release film 926 covering electrodes 920 is selected in a color conspicuous to the user, such that the user will readily determine if the tape has been removed or not.

Examples use circuitry and firmware to stimulate the electrode circuit with a brief, low energy pulse or pulse sequence when patch 100 is initially activated. If patch 100 is activated before it is affixed to the skin, the electrode readiness test will fail. In such a case, the electrode readiness test is repeated, again and again according to timers in the firmware or hardware, until either the timers have all expired or the test passes. The test passes when patch 100 is found to exhibit a circuit performance appropriate to its design. The test fails when patch 100 is not properly prepared, such as not removing the electrode films, or is not yet applied to the skin when the timers have all expired. When the electrode readiness test fails, patch 100 signals to the fob or the smart controller, which in turn informs the user. The electrode readiness test is implemented in a manner which may be undetectable by the user, and to minimize the test's use of battery power.

Removable Paper

In some examples, a removable paper 914 covers the adhesive side of bottom layer 910. Removable paper 914 may be in multiple sections, each to be pulled away by the user when affixing patch 100 to the skin. These removable papers may be in addition to the piece of PET film 926 covering each electrode 920. Therefore, the user must remove all of these pieces to expose a complete, adhesive surface to affix to the skin in examples.

In examples, bottom layer 910 is one complete piece, with one removable paper 914. The user removes all of the removable paper in one motion. In examples, bottom layer 910 is two or more pieces, with two or more removable papers 914. The user removes all of the removable papers. In examples, the single removable paper 914 is designed with a pull tab, so that the user pulls the removable paper off of the bottom layer in a direction at right angle to the long axis of patch 100. This motion reduces the forces experienced by the assembled internal components of patch 100.

In examples, removable paper 914 covers bottom layer 910 and covers all of the PET film sections 926. An adhesive attaches the removable paper top surface to the polyimide tape A skin-facing surface, such that the user pulls the removable paper away from the bottom layer and in one motion removes the PET film pieces from electrodes 920.

Patch 100 can also be made more comfortable by the addition of material between the top layer and the bottom layer, such as cushioning material that can cushion the electrodes and electronic components. The cushioning material may be disposed subjacent to the bottom layer and superjacent to the top layer, in at least a portion of patch 100. A cushioning material may include cellulosic fibers (e.g., wood pulp fibers), other natural fibers, synthetic fibers, woven or nonwoven sheets, scrim netting or other stabilizing structures, superabsorbent material, foams, binder materials, or the like, as well as combinations thereof.

Hydrogel Overlaps Electrode Edges

In some examples, each electrode 920 is covered with hydrogel 926 which conforms to the size of the electrode 920, such that the edge of electrode 920 is exposed to the user's skin when patch 100 is applied to the skin. This edge may abrade or cut the user's skin during the time when patch 100 is affixed to the skin.

In some examples, hydrogel 926 is dimensioned so as to overlap the edges of electrode 920. Hydrogel 926 is placed over electrode 920 with the accuracies of placement used in manufacturing, such that the edges of electrode 920 is always covered with hydrogel 926. This keeps the edge electrode 920 from touching the user's skin. The risk of electrodes 920 from abrading or cutting the user's skin is therefore eliminated.

Several examples are specifically illustrated and/or described herein. However, it will be appreciated that modifications and variations of the disclosed examples are covered by the above teachings and within the purview of the appended claims without departing from the spirit and intended scope of the invention.

What is claimed is:

1. A method of healing damage to a fibrous connective tissue, the method comprising:
affixing a patch externally on a dermis of a user adjacent to a damaged fibrous connective tissue of the user, the patch comprising a flexible substrate, a processor directly coupled to the substrate, and electrodes directly coupled to the substrate; and
activating the patch, the activating comprising generating an electrical stimuli via the electrodes that is directed to the damaged fibrous connective tissue.

2. The method of claim 1, the electrical stimuli comprising a series of pulses with a pattern comprising an intensity and a duration, further comprising adjusting the intensity or the duration of the pattern after each treatment of the damaged fibrous connective tissue.

3. The method of claim 1, the electrical stimuli comprising a series of pules with a pattern, comprising pulse widths of 50-200 microseconds and voltage of 100-500 volts.

4. The method of claim 3, the electrical stimuli causing an improvement in alignment of collagen that forms the damaged fibrous connective tissue.

5. The method of claim 1, the damaged fibrous connective tissue comprising plantar fascia and the electrodes comprising a plurality of positive electrodes and at least one negative electrode, the patch affixed so that the electrodes are placed axially along a path of the plantar fascia.

6. The method of claim 1, the patch comprising one or more sensors that measure biometrics of the user and based on the measurement adjusting the electrical stimuli.

7. The method of claim 1, the patch comprising one or more sensors in communication with a smart controller, the smart controller receiving data from the sensors and using the data to orient the patch relative to the user.

8. The method of claim 1, further comprising:
determining a target charge level;
outputting a series of pulses from the electrodes;
for each pulse outputted, measuring a charge value of the pulse and compare the charge value to the target charge level;
if the charge value is greater than the target charge level, reducing a strength level of a subsequent outputted pulse; and
if the charge value is less than the target charge level, increasing the strength level of a subsequent outputted pulse.

9. The method of claim 8, the determining the target charge level Q_{target} comprises generating an acquisition series of pulses and
$${Q}_{target} = {\sum }_{i = 1}^{T * f} {Q}_{pulse} ( i ) ,$$
where T is a duration of the acquisition series of pulses, f is a frequency of the acquisition series of pulses and Qₚᵤₗₛₑ (i) is a measured charge of each of the acquisition series of pulses.

10. The method of claim 8, the patch further comprising electronic circuitry directly coupled to the substrate and comprising a differential integrator, the charge value of the pulse based on an output of the differential integrator.

11. A fibrous connective tissue damage healing system comprising:
a patch adapted to be externally coupled on a dermis of a user adjacent to a damaged fibrous connective tissue of the user, the patch comprising a flexible substrate, a processor directly coupled to the substrate, and electrodes directly coupled to the substrate; and
the processor adapted to activate the patch, the activating comprising generating an electrical stimuli via the electrodes that is directed to the damaged fibrous connective tissue.

12. The system of claim 11, the electrical stimuli comprising a series of pulses with a pattern comprising an intensity and a duration, further comprising adjusting the intensity or the duration of the pattern after each treatment of the damaged fibrous connective tissue.

13. The system of claim 11, the electrical stimuli comprising a series of pules with a pattern, comprising pulse widths of 50-200 microseconds and voltage of 100-500 volts.

14. The system of claim 13, the electrical stimuli causing an improvement in alignment of collagen that forms the damaged fibrous connective tissue.

15. The system of claim 11, the damaged fibrous connective tissue comprising plantar fascia and the electrodes comprising a plurality of positive electrodes and at least one negative electrode, the patch affixed so that the electrodes are placed axially along a path of the plantar fascia.

16. The system of claim 11, the patch comprising one or more sensors that measure biometrics of the user and based on the measurement adjusting the electrical stimuli.

17. The system of claim 11, the patch comprising one or more sensors in communication with a smart controller, the smart controller receiving data from the sensors and using the data to orient the patch relative to the user.

18. The system of claim 11, the processor further adapted to:
determining a target charge level;
outputting a series of pulses from the electrodes;
for each pulse outputted, measuring a charge value of the pulse and compare the charge value to the target charge level;
if the charge value is greater than the target charge level, reducing a strength level of a subsequent outputted pulse; and
if the charge value is less than the target charge level, increasing the strength level of a subsequent outputted pulse.

19. The system of claim 18, the determining the target charge level Q_{target} comprises generating an acquisition series of pulses and
$${Q}_{target} = {\sum }_{i = 1}^{T * f} {Q}_{pulse} ( i ) ,$$
where T is a duration of the acquisition series of pulses, f is a frequency of the acquisition series of pulses and Qₚᵤₗₛₑ (i) is a measured charge of each of the acquisition series of pulses.

20. The system of claim 18, the patch further comprising electronic circuitry directly coupled to the substrate and comprising a differential integrator, the charge value of the pulse based on an output of the differential integrator.
